(19) United States
(12) Patent Application Publication
(10) Pub. No.: US 2025/0139851 A1
(43) Pub. Date: May 1, 2025

(54) PET IMAGING METHOD, DEVICE, STORAGE MEDIUM, AND PROGRAM PRODUCT

(71) Applicant: Shanghai United Imaging Healthcare Co., Ltd., Shanghai (CN)

(72) Inventors: Liuchun He, Shanghai (CN); Yun Dong, Shanghai (CN)

(21) Appl. No.: 18/928,243

(22) Filed: Oct. 28, 2024

(30) Foreign Application Priority Data
Oct. 26, 2023 (CN) 202311404861.1
Dec. 6, 2023 (CN) 202311669277.9

(51) Int. Cl. G06T 11/00; A61B 6/00; A61B 6/03; G06T 7/00

(52) U.S. Cl. CPC G06T 11/005; A61B 6/037; A61B 6/5282; G06T 7/0012; G06T 2207/10104; G06T 2207/30004; G06T 2210/41; G06T 2211/408; G06T 2211/452

(57) ABSTRACT

The present disclosure relates to a positron emission computed tomography (PET) imaging method, a device, a storage medium, and a product. The method includes: determining a target coincidence event of a to-be-detected object; and performing image reconstruction based on the PET raw data corresponding to the target coincidence event, to obtain a reconstructed image of the to-be-detected object.

### CROSS-REFERENCE TO RELATED APPLICATIONS

This application claims priority to Chinese patent application No. 202311404861.1, filed on Oct. 26, 2023 and Chinese patent application No. 202311669277.9, filed on Dec. 6, 2023, the contents of which are hereby incorporated by reference in their entireties.

### TECHNICAL FIELD

The present disclosure relates to the field of positron emission computed tomography (PET) imaging technologies, and in particular, to a PET imaging method, a device, a storage medium, and a program product.

### BACKGROUND

With the development of a medical imaging technology, PET is used more and more widely. During PET scanning, when two 511 keV gamma (γ) photons are collected by a pair of PET detectors within a determined time window, an event mark is generated, called a coincidence event. According to acquisition of and subsequent processing on the coincidence event, a reconstructed image can be obtained for PET imaging.

### SUMMARY

Based on this, the present disclosure provides a PET imaging method, a device, a storage medium, and a program product.

In a first aspect, the present disclosure provides a positron emission computed tomography (PET) imaging method, which includes: determining a target coincidence event of a to-be-detected object; and performing image reconstruction based on PET raw data corresponding to the target coincidence event, to obtain a reconstructed image of the to-be-detected object.

In an embodiment, determining the target coincidence event of the to-be-detected object includes: determining, based on a first energy window and a second energy window that are asymmetrical to each other, the target coincidence event of the to-be-detected object.

In an embodiment, the first energy window includes a low energy-level discriminator (LLD) threshold and/or a high energy-level discriminator (HLD) threshold, and the second energy window includes a LLD threshold and/or a HLD threshold; and wherein the LLD threshold and/or the HLD threshold of the first energy window is different from the LLD threshold and/or the HLD threshold of the second energy window.

In an embodiment, the first energy window includes a first low energy-level discriminator (LLD) threshold; the second energy window includes a second LLD threshold; and wherein the first LLD is different from the second LLD.

In an embodiment, the first LLD threshold and the second LLD threshold are determined by the following process: acquiring a first low energy-level initial threshold and a second low energy-level initial threshold; adjusting the first low energy-level initial threshold and the second low energy-level initial threshold according to a preset condition, to obtain the first LLD threshold and the second LLD threshold that are different from each other.

In an embodiment, the method further includes: obtaining a scattering correction coefficient; and correcting the target coincidence event of the to-be-detected object according to the scattering correction coefficient.

In an embodiment, the scattering correction coefficient is determined by performing Monte Carlo simulation on a simulated detection object.

In an embodiment, the method further includes: performing, in response to a trigger instruction of an asymmetrical energy window mode, the step of determining the target coincidence event of the to-be-detected object based on the first energy window and the second energy window that are asymmetrical to each other.

In an embodiment, performing image reconstruction based on the PET raw data corresponding to the target coincidence event, to obtain the reconstructed image of the to-be-detected object includes: dividing the PET raw data into at least two data subsets according to a data characteristic of the PET raw data; performing physical correction on the data subsets respectively; and performing image reconstruction based on the corrected data subsets to obtain the reconstructed image of the to-be-detected object.

In an embodiment, the data characteristic of the PET raw data includes one or a combination of the following: energy information of the PET raw data; event information of the PET raw data.

In an embodiment, the data quality rule includes a data information rule, the data information rule includes energy information; and dividing the PET raw data into the at least two data subsets according to the data characteristic of the PET raw data includes: dividing the PET raw data into the at least two data subsets according to energy information of the PET raw data.

In an embodiment, the PET raw data includes first data and second data obtained from a pair of detectors at two ends of the target coincidence event; and dividing the PET raw data into the at least two data subsets according to the energy information of the PET raw data includes: dividing the PET raw data into a first data subset when energies of the first data and the second data are both greater than a preset energy; and dividing the PET raw data into a second data subset when the energy of the first data or the energy of the second data is less than or equal to the preset energy.

In an embodiment, dividing the PET raw data into the at least two data subsets according to the data characteristic of the PET raw data includes: dividing the PET raw data into the at least two data subsets according to whether to be the scattering sequence recovery event.

In an embodiment, the PET raw data includes third data and fourth data obtained from a pair of detectors at two ends of the target coincidence event; and dividing the PET raw data into the at least two data subsets according to whether to be the scattering sequence recovery event includes: dividing the PET raw data into a third data subset when both the third data and the fourth data do not coincide with the scattering sequence recovery event; and dividing the PET raw data into a fourth data subset when the third data or the fourth data coincides with the scattering sequence recovery event.

In an embodiment, performing physical correction on the data subsets respectively includes: performing physical correction on the data subsets respectively by using a delay coincidence window method.

In an embodiment, performing image reconstruction based on the corrected data subsets to obtain the reconstructed image of the to-be-detected object includes: performing image reconstruction based on the corrected the data subsets and according to an image reconstruction iterative algorithm to obtain the reconstructed image of the to-be-detected object.

In an embodiment, performing image reconstruction based on the corrected the data subsets and according to an image reconstruction iterative algorithm to obtain the reconstructed image of the to-be-detected object the includes: performing modeling on each of the corrected data subsets to obtain modeling data of each of the data subsets; performing a joint reconstruction on a combination of the modeled data of the data subsets to obtain the reconstructed image of the to-be-detected object.

In a second aspect, the present disclosure provides a PET imaging method, the method includes: determining a target coincidence event of a to-be-detected object, which includes determining, based on a first energy window and a second energy window that are asymmetrical to each other, the target coincidence event of the to-be-detected object; and performing image reconstruction based on the PET raw data corresponding to the target coincidence event to obtain a reconstructed image of the to-be-detected object, which includes: dividing the PET raw data into at least two data subsets according to a data characteristic of the PET raw data; performing physical correction on the data subsets respectively; and performing image reconstruction based on the corrected data subsets to obtain the reconstructed image of the to-be-detected object.

In a third aspect, the present disclosure provides an electronic device, which includes a memory and a processor, the memory storing a computer program, and the processor, when executing the computer program, implementing the above PET imaging method.

In a fourth aspect, the present disclosure provides a non-transitory computer-readable storage medium, having a computer program stored therein, wherein the above PET imaging method is implemented when the computer program is executed by a processor.

Details of one or more embodiments of the present disclosure are set forth in the following accompanying drawings and descriptions. Other features, objectives, and advantages of the present disclosure become obvious with reference to the specification, the accompanying drawings, and the claims.

### BRIEF DESCRIPTION OF THE DRAWINGS

In order to more clearly illustrate the technical solutions in embodiments of the present disclosure or the related art, the accompanying drawings used in the description of the embodiments or the related art will be briefly introduced below. It is apparent that, the accompanying drawings in the following description are only some embodiments of the present disclosure, and other drawings can be obtained by those of ordinary skill in the art from the provided drawings without creative efforts.

FIG. 1A and FIG. 1B are schematic flowcharts of a PET imaging method according to an embodiment;

FIG. 1C is a structural diagram of a PET system according to an embodiment;

FIG. 1D is a structural diagram of a PET device according to an embodiment;

FIG. 2A is a schematic flowchart of a coincidence event determination method according to an embodiment;

FIG. 2B is a schematic flowchart of a coincidence event determination method according to an embodiment;

FIG. 3 is a schematic flowchart of a step of acquiring a first LLD threshold and a second LLD threshold that are different from each other according to an embodiment;

FIG. 4 is a schematic flowchart of a step of acquiring a target coincidence event according to an embodiment;

FIG. 5 is a schematic diagram of results of changes of noise equivalent counts (NECs) with LLD thresholds according to another embodiment;

FIG. 6 is a schematic structural diagram of a scattering correction pressure test phantom according to an embodiment;

FIG. 7 is a schematic diagram of results of a scattering correction test according to an embodiment;

FIG. 8 is a structural block diagram of a coincidence event determination apparatus according to an embodiment;

FIG. 9A and FIG. 9B are a first flowchart of a PET image reconstruction method according to an exemplary embodiment of the present disclosure;

FIG. 10 is a second flowchart of the PET image reconstruction method according to an exemplary embodiment of the present disclosure;

FIG. 11 is a third flowchart of the PET image reconstruction method according to an exemplary embodiment of the present disclosure;

FIG. 12 is a schematic diagram of an existing PET image reconstruction result;

FIG. 13 is a schematic diagram of an example image reconstruction result of the PET image reconstruction method according to an exemplary embodiment of the present disclosure;

FIG. 14 is a comparison diagram of relative intensity between the example image reconstruction result of the PET image reconstruction method according to an exemplary embodiment of the present disclosure and the existing PET image reconstruction result;

FIG. 15 is a schematic structural diagram of a PET image reconstruction system according to an exemplary embodiment of the present disclosure;

FIG. 16 is a schematic flowchart of a PET imaging method according to an exemplary embodiment of the present disclosure;

FIG. 17 is a diagram of an internal structure of a controller according to an exemplary embodiment of the present disclosure; and

FIG. 18 is a schematic structural diagram of an electronic device according to an exemplary embodiment of the present disclosure.

### DETAILED DESCRIPTION

In order to make the objectives, technical solutions, and advantages of the present disclosure clearer, the present disclosure will be further described below in detail with reference to the accompanying drawings and embodiments. It should be understood that specific embodiments described herein are only intended to interpret the present disclosure and are not intended to limit the present disclosure.

It should be noted that the terms “first”, “second”, “third”, “fourth”, “fifth”, and “sixth” are used for descriptive purposes only, which cannot be construed as indicating or implying a relative importance or implicitly specifying the number of the indicated technical features. Thus, the features defined with “first”, “second”, “third”, “fourth”, “fifth”, and “sixth” may explicitly or implicitly include one or more features.

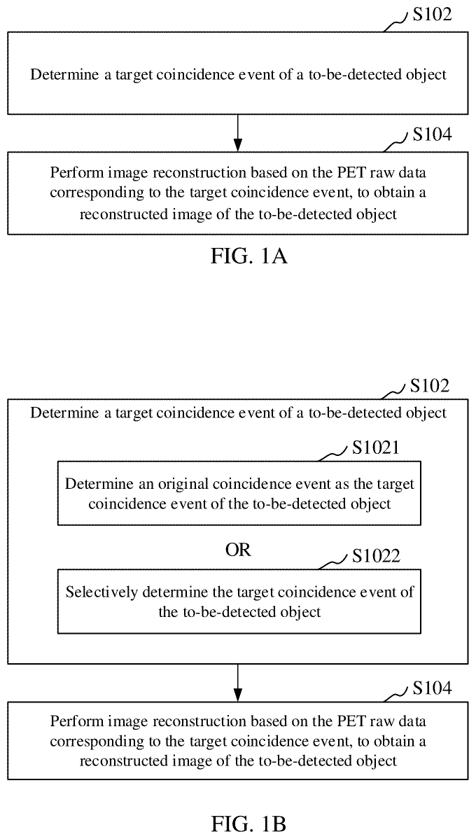

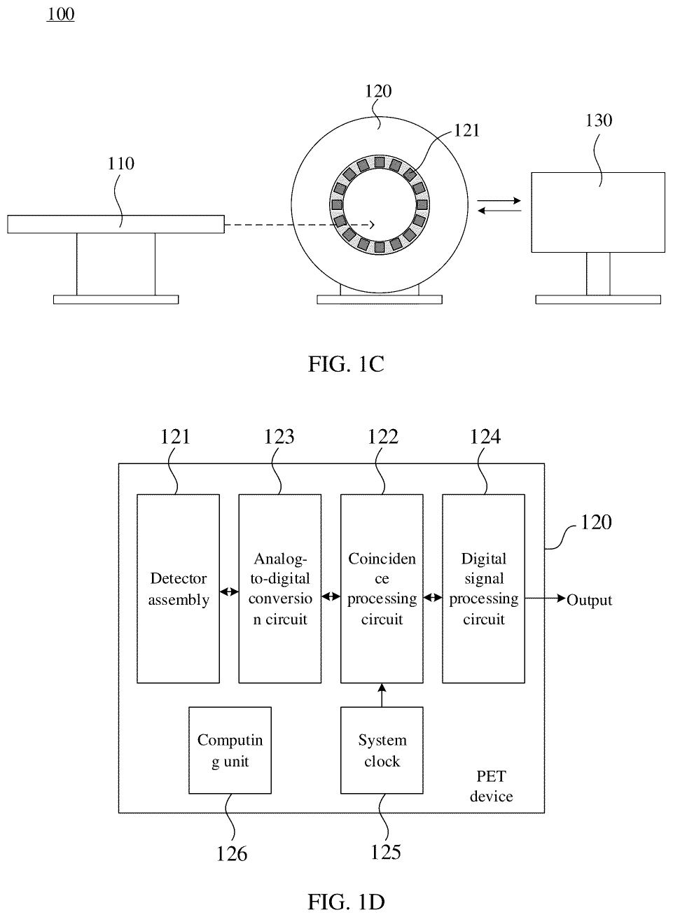

In an embodiment, as shown in FIG. 1A and FIG. 1B, the present disclosure provides a PET imaging method. The method includes steps S102 to S104 below.

In step S102, a target coincidence event of a to-be-detected object is determined.

The target coincidence event of the to-be-detected object determined in step S102 is a coincidence event corresponding to PET raw data to be used to perform image reconstruction.

A process of determining the target coincidence event of the to-be-detected object in step S102 may be performed in various known or improved manners. For example, according to whether an original coincidence event is filtered, the manners may be classified into the following two manners. The original coincidence event refers to all coincidence events detectable in an existing PET device. The original coincidence event may generally include true coincidence events, scattering coincidence events, and random coincidence events. For example, at least one photon scatters once or multiple times with a medium. Such an event is called a scattering coincidence event.

In the first manner, the original coincidence event may not be filtered, and the original coincidence event is directly regarded as the target coincidence event. That is, step S102 may include: step S1021 of determining the original coincidence event to be the target coincidence event of the to-be-detected object.

In the second manner, according to a usage requirement, one or more means may be taken to selectively determine an expected target coincidence event, so that the selectively determined target coincidence event has one or more optimized characteristics compared with the original coincidence event.

For example, in the related art, a response line may be defined according to a coincidence event, to acquire PET raw data to be used to perform reconstruction for PET imaging. However, among the true coincidence events, the scattering coincidence events, and the random coincidence events included in the original coincidence event, only the true coincidence events can accurately reflect positioning information of an annihilation event. Correspondingly, in the second manner, for example, an expected target coincidence event may be selectively determined, so that compared with the original coincidence event, at least part of the scattering coincidence events and/or the random coincidence events are filtered out for the target coincidence event, thereby having a higher proportion of true coincidence events. That is, step S102 may include: step S1022 of selectively determining the target coincidence event of the to-be-detected object. The target coincidence event may thus include at least true coincidence events, and a proportion of the true coincidence events in the target coincidence event is greater than a proportion of the true coincidence events in the original coincidence event.

By way of example but not limitation, a process of selectively determining the target coincidence event in step S1022 may be performed according to a coincidence event determination method that will be discussed later.

In step S104, image reconstruction is performed based on the PET raw data corresponding to the target coincidence event, to obtain a reconstructed image of the to-be-detected object.

The PET raw data may also be called target imaging data, which is data to be used for PET imaging. The PET raw data corresponding to the target coincidence event may contain information related to the target coincidence event, such as the positions of the detectors at both ends of the target coincidence event, the time of flight, and the like.

The reconstructed image may also be called a target image, which may be an image of the to-be-detected object imaged by a PET system.

A process of performing image reconstruction based on the PET raw data, to obtain a reconstructed image of the to-be-detected object in step S104 may be performed in various known or improved manners.

By way of example but not limitation, the process of performing image reconstruction based on the PET raw data, to obtain a reconstructed image of the to-be-detected object in step S104 may be performed according to a PET image reconstruction method that will be discussed later.

It should be noted that during the PET imaging, the coincidence event determination method that will be discussed later in the present disclosure and the PET image reconstruction method that will be discussed later in the present disclosure may be used independently of each other or in combination with each other. In some embodiments, the PET imaging method may include step S102 performed according to the following coincidence event determination method and step S104 performed according to other known or improved methods except the following PET image reconstruction method. In some embodiments, the PET imaging method may include step S102 performed according to other known or improved methods except the following coincidence event determination method and step S104 performed according to the following PET image reconstruction method. In some embodiments, the PET imaging method may include step S102 performed according the following coincidence event determination method and step S104 performed according to the following PET image reconstruction method.

In an exemplary embodiment, the present disclosure further provides a PET imaging apparatus configured to implement the above PET imaging method. The implementation solution to the problem provided in the apparatus is similar to that described in the above method. Therefore, specific limitations in one or more embodiments of the PET imaging apparatus provided below may be obtained with reference to the limitations on the PET imaging method above. Details are not described herein again.

In an exemplary embodiment, a PET imaging apparatus is provided, including: a target coincidence event determination module, and a reconstructed image determination module.

The target coincidence event determination module is configured to determine a target coincidence event of a to-be-detected object.

The reconstructed image determination module is configured to perform image reconstruction based on the PET raw data corresponding to the target coincidence event, to obtain a reconstructed image of the to-be-detected object.

The modules in the PET imaging apparatus above may be implemented entirely or partially by software, hardware, or a combination thereof. The above modules may be built in or independent of a processor of a controller in a hardware form, or may be stored in a memory of the controller in a software form, to facilitate the processor to invoke and perform operations corresponding to the above modules.

In one of the embodiments, as shown in FIGS. 1C and 1D, the present disclosure provides a PET system 100 for implementing any of the PET imaging methods of the present disclosure. The PET system 100 may include a bed 110, a PET device 120, and a computing device 130.

The PET device 120 is configured to detect a to-be-detected object positioned on the bed 110 and output PET data containing information related to coincidence events to the computing device.

The PET device 120 may include a detector assembly 121. The detector assembly 121 may have a structure of a detector ring. The detector ring is distributed with a plurality of detector crystals (which may correspond to the detector described below), and the plurality of detector crystals are preferably arranged in an array. The PET device 120 may also include a coincidence processing circuit 122, which is configured to receive signals output from the detector assembly 121 and output an coincidence event when the received signals satisfy a coincidence time window and a coincidence energy window. The PET device 120, for example, may further include an analog-to-digital conversion circuit 123 connected between the detector assembly 121 and the coincidence processing circuit 122, a digital signal processing circuit 124 connected to the output end of the coincidence processing circuit 122, a system clock 125, and the like. In addition, the PET device 120 may further include a computing unit 126 configured to process detection data within the PET device 120.

The computing device 130 is configured to perform processing based on the received PET data to generate a PET image.

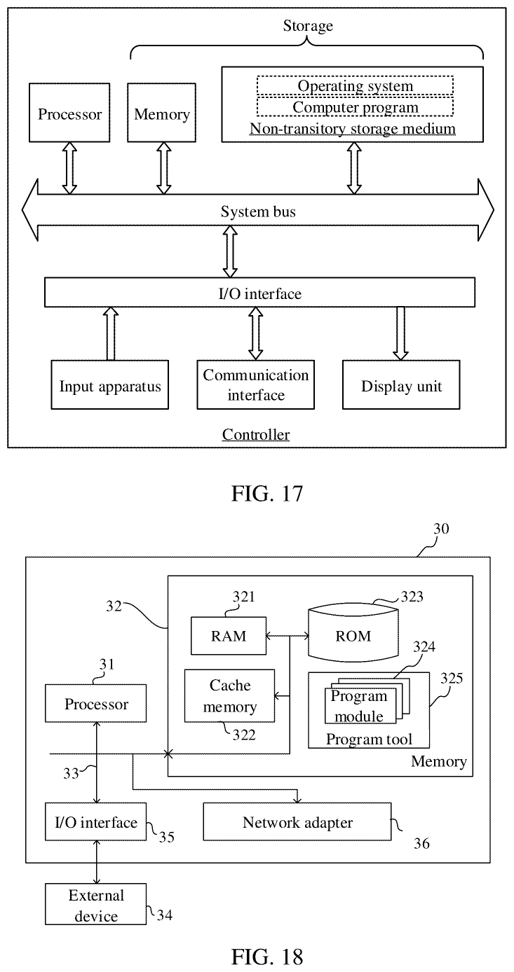

The computing device 130 is an equipment with computing and processing capability. For example, it may be implemented by a computer terminal and/or a server. When the computing device 130 includes a terminal, the terminal, for example, may be an operation control console. The computing device 130 may be implemented as including a controller as shown in FIG. 17 or an electronic device as shown in FIG. 18. The computing device 130 may also include or be connected to a display for displaying data related to PET imaging, such as a reconstructed image.

In a PET detection system, a true coincidence event expected by a PET detector is a coincidence event generated from annihilation of β+ electrons (positively charged beta particles) and not scattering before entering the PET detector. Gamma photons of the true coincidence event have an energy of 511 keV before entering the PET detector, but detection efficiency of the detector for 511 keV rays is not 100%. There is also a difference between all-energy peak detection efficiency and all-energy spectrum detection efficiency. The all-energy peak detection efficiency refers to detection efficiency with which the PET detector captures energies of all the 511 keV rays, while the all-energy spectrum detection efficiency refers to efficiency with which the PET detector interacts with the 511 keV rays to capture a certain amount of energy. Since some rays only undergo a small amount of Compton scattering in the PET detector and only deposit part of the energy, there is generally a >20% difference between the two types of detection efficiency. Since the PET detection system detects coincidence events, the difference between the all-energy peak detection efficiency and the all-energy spectrum detection efficiency for the coincidence events may generally reach 40%.

Although lowering an LLD threshold of a system energy window can improve system sensitivity, scattering and random events may be increased, resulting in a decrease in a noise equivalent count rate (NECR). On the contrary, for a system with high-level resolution, increasing the LLD threshold may increase a NECR of the system, but sensitivity of the system may also be reduced.

In the related art, a symmetrical energy window setting may be used, that is, when energies at two ends of a coincidence are both greater than the LLD threshold, the coincidence event is received: Eₐ>E_{LLD} ∩E_{b}>E_{LLD}, where Eₐ and E_{b} denote the energies at the two ends of the coincidence respectively, and E_{LLD} denotes the LLD threshold. In this way, balance between system sensitivity and NECR performance can be pursued, but the symmetrical energy window setting still has the problem of low accuracy in determining a coincident event.

In an embodiment, the present disclosure provides a coincident event determination method, which may be used to perform step S102, that is, the step of determining a target coincidence event of a to-be-detected object. In other words, step S102 may include the steps of the coincident event determination method as described below.

In the embodiments of the present disclosure, for example, the method is applied to a terminal (e.g., a computer). It may be understood that the method is also applicable to a server, or is applicable to a system including a terminal and a server and is implemented through interaction between the terminal and the server.

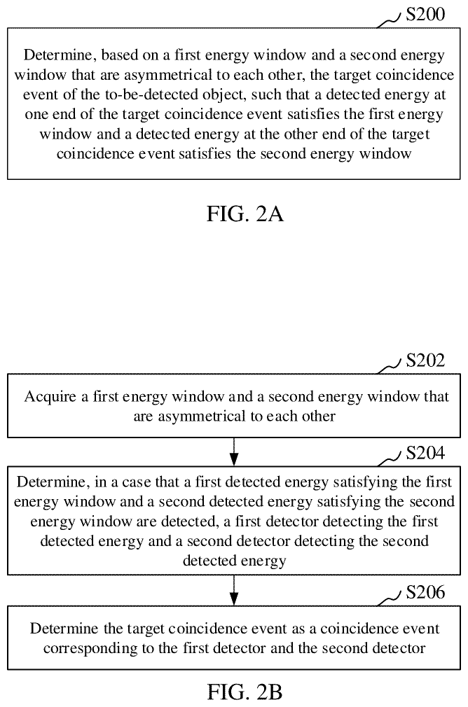

In an exemplary embodiment, as shown in FIG. 2A, the coincident event determination method may include step S200:

In step S200, based on a first energy window and a second energy window that are asymmetrical to each other, the target coincidence event of the to-be-detected object is determined such that a detected energy at one end of the target coincidence event satisfies the first energy window and a detected energy at the other end of the target coincidence event satisfies the second energy window.

The target coincidence event is defined by a first detected event and a second detected event located at two ends of the target coincidence event. The first detected event is detected by a first detector, and the first detected energy of the first detected event meets the first energy window. The second detected event is detected by a second detector, and the second detected energy of the second detected event meets the second energy window.

In some embodiments, the target coincidence event of the to-be-detected object is further determined based on a preset time window. The target coincidence event of the to-be-detected object is determined such that a first detection time of the first detected energy and a second detection time of the second detected energy are fall within a same preset time window.

The above coincident event determination method of this disclosure may be implemented by software, by hardware, or by a combination of software and hardware. The method may be performed partially or entirely by a PET device, partially or entirely by a computing device, or performed by both a PET device and a computing device in cooperation with each other.

Exemplarily, the above step S200 may be performed at least in part based on the hardware by a PET device. Modifications may be made to the structure of the circuits inside the PET device to configure it to at least partially implement the above method. For example, the coincidence processing circuit in the PET device may be configured in hardware structure so that it can acquire a first detected event with a first detected energy satisfying the first energy window and a second detected event with a second detected energy satisfying the second energy window asymmetric to the first energy window, within a preset time window, and may output acquired coincidence event containing the first detection event and the second detection event as the target coincidence event. After that, the computing device may receive the target coincidence event from the PET device, and thus the PET data corresponding to the target coincidence event is obtained. The computing device may perform reconstruction on the PET data corresponding to the target coincidence event to obtain the reconstructed image.

Exemplarily, the above step S200 may be performed at least in part based on the software by the computing device. In such an example, the PET device may have a configuration similar to the prior art that uses a symmetric energy window to detect coincidence events. Based on a symmetric energy window, the coincidence processing circuit of the PET device detects and outputs basic coincidence events. The base coincidence events output by the PET device may then be received by the computing device. The computing device selects, from the received basic coincidence events, target coincidence events meeting the first energy window and the second energy window. That is, when the first detected energy at one end of a received basic coincidence event meets the first energy window, and the second detected energy at the other end of the received basic coincidence event meets the second energy window, the computing device determine the received basic coincidence event as the target coincidence event. After that, the computing device may perform reconstruction on the PET data corresponding to the target coincidence event to obtain the reconstructed image.

In an exemplary embodiment, as shown in FIG. 2B, the coincident event determination method includes steps S202 to S206 below.

In step S202, a first energy window and a second energy window that are asymmetrical are acquired.

The first energy window and the second energy window may both be preset energy windows.

The first energy window and the second energy window may be provided as two energy windows asymmetrical to each other. The asymmetry between the two energy windows means that the allowable value ranges of the two energy windows are not exactly the same.

Each of the energy windows may include an LLD threshold and a high energy-level discriminator (HLD) threshold. For example, the energy falls between the LLD threshold and the HLD threshold may be regarded as satisfying the energy window. The two energy windows being asymmetrical to each other may means that the two energy windows have different LLD thresholds and/or HLD thresholds. For example, if an LLD threshold of the first energy window and an LLD threshold of the second energy window are different, then the first energy window and the second energy window are two energy windows being asymmetrical to each other.

In the present disclosure, as an example, the LLD threshold of the first energy window is different from the LLD threshold of the second energy window, and the HLD threshold of the first energy window can be the same as the HLD threshold of the second energy window.

Exemplarily, the terminal may acquire a first energy window and a second energy window that are asymmetrical and preset by a user. For example, the terminal may determine an HLD threshold and an LLD threshold of the first energy window and an HLD threshold and an LLD threshold of the second energy window. The LLD threshold of the first energy window and the LLD threshold of the second energy window are different, which are taken as the first energy window and the second energy window that are asymmetrical.

Optionally, E_{LLD}1>E_{LLD2} may be set, where E_{LLD}, denotes the LLD threshold of the first energy window, and E_{LLD2} denotes the LLD threshold of the second energy window.

It is understood that in actual operation, the steps of acquiring the first energy window and the second energy window are not necessary. For example, the first energy window and the second energy window can be determined in advance. The first and second energy windows can be pre-configured as configuration parameters or structure features in a terminal or server or detector device (e.g. a circuit structure thereof). When determining the target coincidence event, it can directly making the determination based on the first energy window and the second energy window.

In step S204, in a case that a first detected energy satisfying the first energy window and a second detected energy satisfying the second energy window are detected, a first detector detecting the first detected energy and a second detector detecting the second detected energy are determined.

The first detected energy may be a detected energy within the first energy window. The second detected energy may be a detected energy within the second energy window. The first detector may be a detector that detects the first detected energy. The energy detected by the first detector satisfies the first energy window. The second detector may be a detector that detects the second detected energy. The energy detected by the second detector satisfies the second energy window.

It may be understood that the terms “first”, “second”, and the like used in the present disclosure may be used herein to describe various elements, but these elements are not limited by these terms. These terms are only used to distinguish one element from another element. For example, without departing from the scope of the present disclosure, at a current moment, if an energy detected by a detector satisfies the second energy window, the detector may be taken as the second detector. At another moment, if an energy detected by a detector satisfies the first energy window, the detector may be taken as the first detector. At a same moment, the first detector and the second detector are both detectors, but they are not the same detector.

Exemplarily, the first detected energy satisfying the first energy window and the second detected energy satisfying the second energy window may be determined within a preset time window. The time window may be a preset time range. For example, within the preset time range, the first detector detects the first detected energy satisfying the first energy window, and the second detector detects the second detected energy satisfying the second energy window. A time difference between the first detected energy detected by the first detector and the second detected energy detected by the second detector is within the preset time range.

If the first detected energy satisfying the first energy window and the second detected energy satisfying the second energy window are detected in the preset time window, the first detector detecting the first detected energy and the second detector detecting the second detected energy may be determined. Further, the target coincidence event may be determined as a coincidence event corresponding to the first detected energy detected by the first detector and the second detected energy detected by the second detector.

In S206, the target coincidence event is determined as a coincidence event corresponding to the first detector and the second detector.

The target coincidence event may be a coincidence event to be used for imaging of the PET detection system. For example, the target coincidence event may include at least true coincidence events. It may be understood that in this embodiment, it is desired that the detected target coincidence event may include as many true coincidence events as possible, and at the same time, scattering coincidence events and random coincidence events may be filtered out as much as possible, so as to increase a proportion of the true coincidence events in the determined target coincidence event.

Exemplarily, if first detected energy detected by a first detector satisfies the first energy window and second detected energy detected by a second detector satisfies the second energy window in the preset time window, a coincidence event including a first detected event with the first detected energy by the first detector and a second detected event with the second detected energy by the second detector may be taken as the target coincidence event.

Optionally, two different low-level thresholds E_{LLD1} and E_{LLD2} may be set, and E_{LLD1}>E_{LLD2}. An event greater than E_{LLD1} represents an energy spectrum region corresponding to the all-energy peak detection efficiency, and E_{LLD1} to E_{LLD2} regions are energy expansion regions to improve system sensitivity, where E_{LLD1} to E_{LLD2} means that, within a range from E_{LLD1} to E_{LLD2}, when the detected energies at two ends of a coincidence event are both greater than E_{LLD2} and the deposition energy at one end is greater than E_{LLD1}, the first detector and the second detector may be determined according to the following expression, and the target coincidence event may be further determined:

$$( {E}_{a} > {E}_{L L D 1} ⋂ {E}_{b} > {E}_{L L D 2} ) ⋃ ( {E}_{a} > {E}_{L L D 2} ⋂ {E}_{b} > {E}_{L L D 1} )$$

where E_{LLD1} denotes the LLD threshold of the first energy window, and E_{LLD2} denotes the LLD threshold of the second energy window. When E_{b}>E_{LLD2}, Eₐ denotes the first detected energy, and E_{b} denotes the second detected energy. When Eₐ>E_{LLD2}, E_{b} denotes the first detected energy, and Eₐ denotes the second detected energy.

The above expression may represent that the target coincidence event may be determined when Eₐ>E_{LLD1}∩E_{b}>E_{LLD2} or Eₐ>E_{LLD2}∩E_{b}>E_{LLD1} is satisfied.

When Eₐ>E_{LLD1}∩E_{b}>E_{LLD2} is satisfied, a detector detecting an energy Eₐ is the first detector, a detector detecting an energy E_{b} is the second detector, and a coincidence event jointly corresponding to Eₐ and E_{b} may be taken as the target coincidence event, where Eₐ>E_{LLD1}∩E_{b}>E_{LLD2} means that the energy Eₐ>E_{LLD1} is satisfied and the energy E_{b}>E_{LLD2} is satisfied. When Eₐ>E_{LLD2}∩E_{b}>E_{LLD1} is satisfied, a detector detecting E_{b} is the first detector, a detector detecting Eₐ is the second detector, and a coincidence event jointly corresponding to Eₐ and E_{b} may be taken as the target coincidence event, where Eₐ>E_{LLD2}∩E_{b}>E_{LLD1} means that the energy Eₐ>E_{LLD2} is satisfied and the energy E_{b}>E_{LLD1} is satisfied.

In this embodiment, by acquiring a first energy window and a second energy window that are asymmetrical and determining, in a case that a first detected energy satisfying the first energy window and a second detected energy satisfying the second energy window are detected, a first detector detecting the first detected energy and a second detector detecting the second detected energy, a target coincidence event can be determined as being a coincidence event between the first detector and the second detector. In this embodiment, by arranging the first energy window and the second energy window that are asymmetrical, coincidence events can be determined by expanding an energy window, and values with lower energies can be reduced, so as to prevent inaccuracy of the coincidence events. Further, true coincidence events can have a higher proportion in the determined target coincidence event, thereby improving detection efficiency of determination of the true coincidence events.

In an exemplary embodiment, the first energy window includes a first LLD threshold, and the second energy window includes a second LLD threshold.

Acquiring the first energy window and the second energy window that are asymmetrical in step S202 includes:
acquiring the first LLD threshold and the second LLD threshold that are different from each other;
obtaining the first energy window based on the first LLD threshold; and
obtaining the second energy window based on the second LLD threshold.

The first LLD threshold may be a lower bound threshold of the first energy window. The second LLD threshold may be a lower bound threshold of the second energy window.

Exemplarily, the first LLD threshold and the second LLD threshold may be acquired, and the first LLD threshold and the second LLD threshold are determined to be thresholds with different values. The first LLD threshold and the second LLD threshold may be determined through multiple iterative updates. For example, system sensitivity and a NECR of the PET detection system in this state may be determined by receiving coincidence events according to multiple different first LLD thresholds and second LLD thresholds, and the first LLD threshold and the second LLD threshold may be adjusted according to the system sensitivity and the NECR of the PET detection system.

The first energy window may be determined according to the first LLD threshold, and the second energy window may be determined according to the second LLD threshold. For example, the first energy window and the second energy window may be determined respectively according to the first LLD threshold and the second LLD threshold that are different from each other, and the HLD threshold of the first energy window and the HLD threshold of the second energy window may be the same. In this way, the first energy window and the second energy window that are asymmetrical can be determined.

Optionally, a relationship between the first LLD threshold and the second LLD threshold is as follows: E_{LLD1}>E_{LLD2}, where E_{LLD1} denotes the first LLD threshold, and E_{LLD2} denotes the second LLD threshold. A coincidence event with a detected energy greater than E_{LLD1} represents an energy spectrum region corresponding to the full-energy peak detection efficiency, E_{LLD1} to E_{LLD2} is an energy expansion region, so as to receive more coincidence events to improve system sensitivity, and detection energies less than E_{LLD2} can be discarded, to ensure that data quality of the system is not lowered.

In this embodiment, by acquiring different first LLD thresholds and second LLD thresholds, the first energy window can be obtained based on the first LLD threshold, and the second energy window can be obtained based on the second LLD threshold. In this way, the energy window can be expanded to receive more coincidence events, and smaller detection energies can be discarded to limit scattering coincidence events and ensure accuracy of received true coincidence events, thereby improving accuracy of the determined target coincidence event and improving detection efficiency of the true coincidence events.

In an exemplary embodiment, detecting the first detected energy satisfying the first energy window and the second detected energy satisfying the second energy window includes: taking a detected energy greater than the first LLD threshold as the first detected energy; and taking a detected energy greater than the second LLD threshold as the second detected energy.

Exemplarily, the detected energy greater than the first LLD threshold may be taken as the first detected energy satisfying the first energy window, and the detected energy greater than the second LLD threshold may be taken as the second detected energy satisfying the second energy window. Further, the target coincidence event may be determined according to the first detected energy and the second detected energy.

Optionally, the first detected energy and the second detected energy are determined according to the following expression (manner):

$$\begin{matrix}{E}_{L L D 1} > {E}_{L L D 2} \\ ( {E}_{a} > {E}_{L L D 1} ⋂ {E}_{b} > {E}_{L L D 2} ) ⋃ ( {E}_{a} > {E}_{L L D 2} ⋂ {E}_{b} > {E}_{L L D 1} )\end{matrix}$$

where E_{LLD1} denotes the first LLD, and E_{LLD2} denotes the second LLD. When E_{b}>E_{LLD2}, Eₐ denotes the first detected energy, and E_{b} denotes the second detected energy. When Eₐ>E_{LLD2}, E_{b} denotes the first detected energy, and Eₐ denotes the second detected energy.

In this embodiment, the detected energy greater than the first LLD threshold is taken as the first detected energy, and the detected energy greater than the second LLD threshold is taken as the second detected energy. In this way, the first detected energy and the second detected energy can be accurately determined, which can further help determine the coincidence event corresponding to the first detected energy and the second detected energy as the target coincidence event, thereby improving accuracy of the target coincidence event, increasing a proportion of the true coincidence events in the determined target coincidence event, and improving detection efficiency of the true coincidence events.

In an exemplary embodiment, the first LLD threshold and the second LLD threshold are determined by the following process:
acquiring a first low energy-level initial threshold and a second low energy-level initial threshold; where, the first low energy-level initial threshold and a second low energy-level initial threshold may be the same, or may be different from each other;
adjusting the first low energy-level initial threshold and the second low energy-level initial threshold according to a preset condition, to obtain the first LLD threshold and the second LLD threshold that are different from each other.

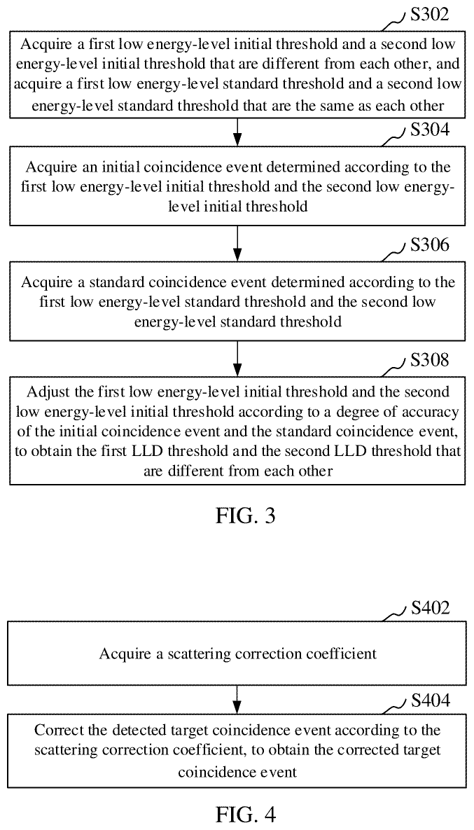

In an exemplary embodiment, as shown in FIG. 3, acquiring the first LLD threshold and the second LLD threshold that are different from each other includes S302 to S308.

In S302, a first low energy-level initial threshold and a second low energy-level initial threshold that are different from each other are acquired, and a first low energy-level standard threshold and a second low energy-level standard threshold that are the same as each other are acquired.

The first low energy-level initial threshold may be an initial first LLD threshold, which may be a first LLD threshold originally set by the user. The second low energy-level initial threshold may be an initial second LLD threshold, which may be a second LLD threshold originally set by the user. The first low energy-level standard threshold and the second low energy-level standard threshold may be LLD thresholds included in symmetrical energy windows.

Exemplarily, two asymmetrical initial energy windows may be determined, and the two initial energy windows respectively include a first low energy-level initial threshold and a second low energy-level initial threshold that are different from each other. The first low energy-level standard threshold and the second low energy-level standard threshold that are the same as each other may be acquired. For example, LLD thresholds corresponding to the symmetrical energy windows may be determined as the first low energy-level standard threshold and the second low energy-level standard threshold that are the same as each other.

In S304, an initial coincidence event determined according to the first low energy-level initial threshold and the second low energy-level initial threshold are acquired.

The initial coincidence event may be a true coincidence event determined according to the first low energy-level initial threshold and the second low energy-level initial threshold that are different from each other.

The initial coincidence event may be determined according to the first low energy-level initial threshold and the second low energy-level initial threshold. For example, a detector that detects a detected energy greater than the first low energy-level initial threshold and a detector that detects a detected energy greater than the second low energy-level initial threshold may be determined, and the coincidence event with two ends corresponding to the above two detectors is regarded as the initial coincidence event.

In S306, a standard coincidence event determined according to the first low energy-level standard threshold and the second low energy-level standard threshold is acquired.

The standard coincidence event may be a true coincidence event determined according to symmetrical energy windows.

Exemplarily, the standard coincidence event may be determined according to the first low energy-level standard threshold and the second low energy-level standard threshold that are the same in the symmetrical energy windows. For example, two detectors that detect energies greater than the LLD threshold of the symmetrical energy windows may be determined, and a coincidence event with its two ends corresponding to the two detectors is taken as the standard coincidence event. As an example, the PET detector uses a symmetrical energy window setting, that is, when energies detected by the PET detector are all greater than a threshold, the coincidence event is received. The standard coincidence event may be determined according to the following expression:

$${E}_{a} > {E}_{L L D} ⋂ {E}_{b} > {E}_{L L D}$$

where E_{LLD} denote the first low energy-level standard threshold and the second low energy-level standard threshold that are the same, i.e., the LLD threshold of the symmetrical energy windows. In the case of Eₐ>E_{LLD}∩E_{b}>E_{LLD}, a detector detecting Eₐ and a detector detecting E_{b} are determined, and a coincidence event corresponding to Eₐ and E_{b} determined by the two detectors is taken as a standard coincidence event. The standard coincidence event may be a coincidence event of the PET detector generally determined in the prior art.

In S308, the first low energy-level initial threshold and the second low energy-level initial threshold are adjusted according to a degree of accuracy of the initial coincidence event and the standard coincidence event, to obtain the first LLD threshold and the second LLD threshold that are different from each other.

The degree of accuracy of the initial coincidence event may be determined according to system sensitivity and a NECR of the PET detection system.

Exemplarily, in a case that the initial coincidence event is received, the sensitivity and the NECR of the PET detection system may be determined as the degree of accuracy of the initial coincidence event. In a case that the standard coincidence event is received, the sensitivity and the NECR of the PET detection system may be determined as a degree of accuracy of the standard coincidence event. Further, the first low energy-level initial threshold and the second low energy-level initial threshold may be adjusted according to the degree of accuracy of the initial coincidence event and the degree of accuracy of the standard coincidence event, to obtain the target coincidence event with better accuracy. That is, the first low energy-level initial threshold and the second low energy-level initial threshold may be adjusted according to the sensitivity and the NECR of the PET detection system, so that the sensitivity and the NECR of the PET detection system meet a preset requirement in the case of an adjusted first LLD threshold and an adjusted second LLD threshold. For example, compared with receiving an existing standard coincidence event, sensitivity of PET detection can be improved and a better NECR can be maintained under a setting that the first LLD threshold and the second LLD threshold are different from each other.

In this embodiment, by determining an initial coincidence event determined according to the first low energy-level initial threshold and the second low energy-level initial threshold and determining a standard coincidence event determined according to the first low energy-level standard threshold and the second low energy-level standard threshold, the first low energy-level initial threshold and the second low energy-level initial threshold can be adjusted according to a degree of accuracy of the initial coincidence event and the standard coincidence event, to obtain the first LLD threshold and the second LLD threshold that are different from each other, so that asymmetrical energy windows can be determined accurately, thereby improving accuracy of the target coincidence event and improving detection efficiency of true coincidence events.

In an exemplary embodiment, determining the target coincidence event as a coincidence event corresponding to the first detector and the second detector includes:
acquiring the first detector correspondingly detecting the first detected energy as a target first detector;
acquiring a first detection time at which the first detected energy is detected, and determining the second detector that detects the second detected energy at a second detection time as a target second detector; the first detection time and the second detection time being times within a preset time window; and
obtaining the target coincidence event according to coincidence events detected by the target first detector and the target second detector.

The target first detector may be a detector that detects the first detected energy satisfying the first energy window. The first detection time may be a time at which the target first detector detects the first detected energy. The second detection time may be a time within a preset time window with the first detection time. For example, a time difference between the second detection time and the first detection time is within the preset time window. The target second detector may be a detector that detects the second detected energy at the second detection time.

Exemplarily, the first detected energy satisfying the first energy window may be detected, and the first detection time at which the first detected energy is detected may be acquired. It may be determined whether the second detected energy satisfying the second energy window is detected within the preset time window, and a time at which the second detected energy is detected is the second detection time. The target first detector detecting the first detected energy at the first detection time may be determined, and the target second detector detecting the second detected energy at the second detection time may be determined. Further, detection events of the target first detector and the target second detector within the above preset time window may be taken as the target coincidence event.

In this embodiment, the target first detector correspondingly detecting the first detected energy at the first detection time is acquired, and the target second detector detecting the second detected energy at the second detection time which is within the preset time window with the first detection time is determined, so that the target coincidence event can be obtained according to coincidence events detected by the target first detector and the target second detector. Through the above process, the proportion of true coincidence events in the determined target coincidence event can be increased by at least partial filtering, thereby improving detection efficiency of the true coincidence events.

In an exemplary embodiment, as shown in FIG. 4, determining the target coincidence event as a coincidence event corresponding to the first detector and the second detector includes the following steps.

In S402, a scattering correction coefficient is acquired.

For example, the scattering correction coefficient may be determined in advance. The scattering correction coefficient may be determined by performing Monte Carlo simulation on a preset simulated detection object.

In S404, the coincidence event as determined is corrected according to the scattering correction coefficient, to obtain the corrected target coincidence event.

The simulated detection object may be a simulated object for detection in scattering correction. The scattering correction coefficient may be a parameter for scattering correction. Scattering correction may be used to correct scattering coincidence events.

Exemplarily, Monte Carlo simulation may be performed on the preset simulated detection object according to preset detection parameters of the PET detector, to obtain the scattering correction coefficient. A to-be-detected object is detected according to the detection parameters of the PET detector, to obtain a coincidence event, and a coincidence event as detected may be corrected by using the scattering correction coefficient, to obtain the corrected target coincidence event.

In this embodiment, by performing Monte Carlo simulation on a preset simulated detection object to obtain a scattering correction coefficient and correcting the coincidence event as detected according to the scattering correction coefficient, a more accurate corrected target coincidence event can be obtained, thereby further improving accuracy of the target coincidence event and improving detection efficiency of true coincidence events.

In an exemplary embodiment, in response to a trigger instruction of an asymmetric energy window mode, the imaging mode can be switched to the asymmetric energy window mode, and step S200 can be executed in the asymmetric energy window mode.

In an exemplary embodiment, acquiring the first energy window and the second energy window that are asymmetrical includes:
acquiring, in response to a trigger instruction of an asymmetrical energy window mode, the asymmetrical energy window mode; and
acquiring the first energy window and the second energy window that are asymmetrical in a case that an imaging mode is the asymmetrical energy window mode.

The asymmetrical energy window mode may be a mode that triggers a reference to an asymmetrical energy window. The imaging mode may be an imaging mode of the PET system.

Exemplarily, the asymmetrical energy window mode may be taken as a special configuration, which may be enabled manually by the user or adaptively enabled in a specific scanning scenario. The trigger instruction of the asymmetrical energy window mode may be a user-triggered instruction or an adaptive triggering instruction. In response to the trigger instruction of the asymmetrical energy window mode, the asymmetrical energy window mode is enabled. The first energy window and the second energy window that are asymmetrical are acquired in a case that the asymmetrical energy window mode is enabled. For example, in a case that the asymmetrical energy window mode is enabled, the terminal may determine an HLD threshold and an LLD threshold of the first energy window and an HLD threshold and an LLD threshold of the second energy window. The LLD threshold of the first energy window and the LLD threshold of the second energy window are different, which are taken as the first energy window and the second energy window that are asymmetrical.

In this embodiment, by acquiring, in response to a trigger instruction of an asymmetrical energy window mode, the asymmetrical energy window mode, the first energy window and the second energy window that are asymmetrical can be acquired in a case that an imaging mode is the asymmetrical energy window mode, thereby expanding the energy windows to determine coincidence events and improving accuracy of the coincidence events.

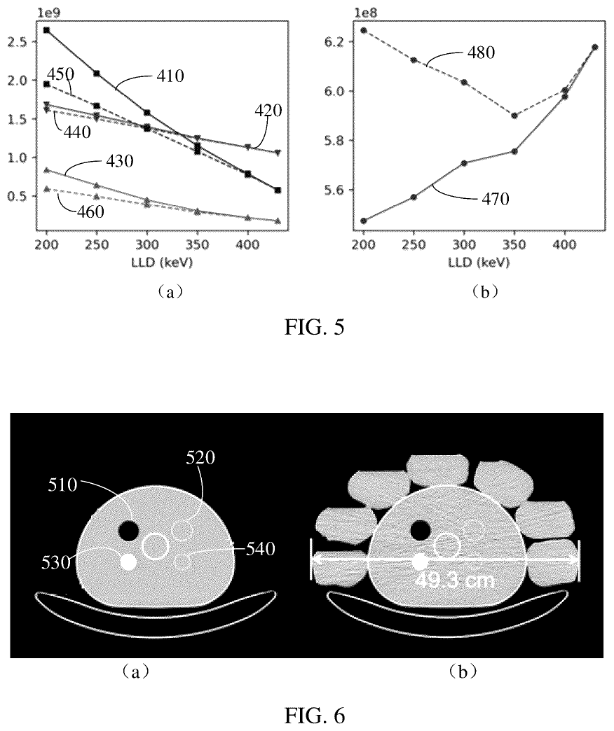

In an exemplary embodiment, in a PET detector with an axial length of 150 cm, a low-activity phantom experiment is performed to observe counts of true coincidence events, scattering coincidence events, and random coincidence events and changes in NECs of the PET system with LLD thresholds of the energy window. Results are shown in FIG. 5. As shown in (a) in FIG. 5, the abscissa is LLD threshold, the ordinate is quantity. Curve 410 is a count of true coincidence events determined by standard symmetrical energy windows, Curve 420 is a count of scattering coincidence events determined by the standard symmetrical energy windows, Curve 430 is a count of random coincidence events determined by the standard symmetrical energy windows, Curve 440 is a count of true coincidence events determined by the asymmetrical energy windows according to the present disclosure, Curve 450 is a count of scattering coincidence events determined by the asymmetrical energy windows according to the present disclosure, and Curve 460 is a count of random coincidence events determined by the asymmetrical energy windows according to the present disclosure. As shown in (a) in FIG. 5 below, according to the coincidence event determination method in the present disclosure, in a case that the LLD threshold is reduced, the system can significantly reduce introduction of the scattering coincidence events and the random coincidence events and try to prevent reduction of the true coincidence events, thereby obtaining a result that a higher NEC is maintained. As shown in (b) in FIG. 5, the abscissa is LLD threshold, and the ordinate is NEC. Curve 470 is a NEC corresponding to the standard symmetrical energy windows, and Curve 480 is a NEC corresponding to the asymmetrical energy windows according to the present disclosure. By use of the solution of the standard symmetrical energy windows, even if the LLD threshold is reduced to 200 keV, the true coincidence events of the PET system can increase by 59.2%, but the NEC may decrease by 11.4%. By use of the coincidence event determination method according to the present disclosure, when E_{LLD1}=430 keV, E_{LLD2}=200 keV, the sensitivity of the PET system increases by 52.4%, and the NEC increases by 1.1%.

In an exemplary embodiment, to verify that Monte Carlo scattering correction can handle the asymmetrical energy windows of the coincidence event determination method according to the present disclosure, a scattering correction pressure test phantom is designed, as shown in FIG. 6. A central lung region of the phantom is filled with a high-activity liquid source, and a contrast with the background is 50:1. In (a) in FIG. 6, 6 hot balls in an original scattering correction pressure test phantom is changed to 4 passive cold balls with different densities, namely a 37-mm air ball 510, a 37-mm water ball 520, a 28-mm salt water ball 530, and a 28-mm water ball 540. It is analyzed through experiments whether Monte Carlo scattering correction can handle multiple scattering events caused by the expanded asymmetrical energy windows. As shown in (b) in FIG. 6, on the basis of normal scanning, a water bag is wrapped around the phantom to increase a proportion of scattering events and increase difficulty of scattering correction.

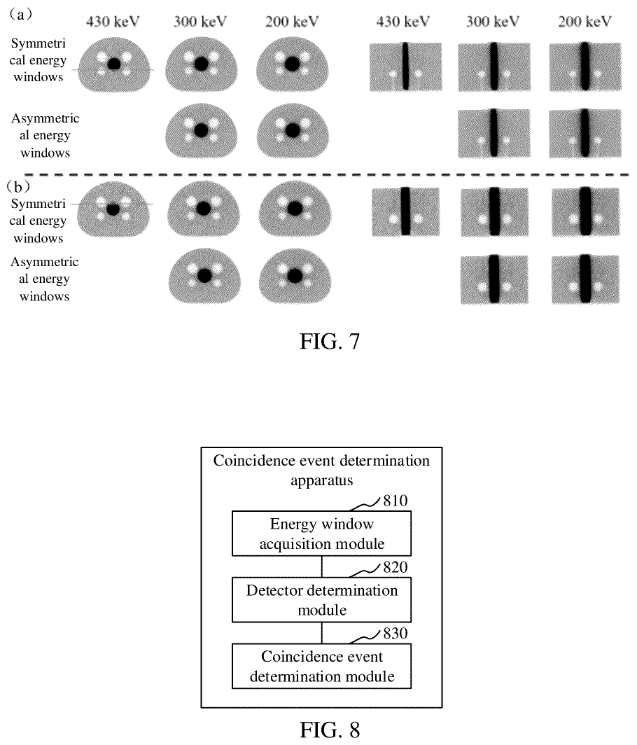

Results are shown in FIG. 7. Monte Carlo scattering correction can handle multiple scattering events caused by the asymmetrical energy windows expanded according to the coincidence event determination method in the present disclosure. (a) and (b) in FIG. 7 correspond to two scanning scenarios (a) and (b) in FIG. 6 respectively.

In some specific embodiments, the coincidence event determination method in the present disclosure can expand the energy window and discard coincidence events with lower energies. In the implementation solution, the logic can be implemented directly by hardware. Alternatively, the coincidence events with lower energies may generally be offline processed and discarded after the energy window is expanded.

It should be understood that, although the steps in the flowchart above are shown in sequence as indicated by the arrows, the steps are not necessarily performed in the order indicated by the arrows. Unless otherwise clearly specified herein, the steps are performed without any strict sequence limitation, and may be performed in other orders. In addition, at least some steps in the flowchart above may include a plurality of steps or a plurality of stages, and such steps or stages are not necessarily performed at a same moment, and may be performed at different moments. The steps or stages are not necessarily performed in sequence, and the steps or stages and at least some of other steps or steps or stages of other steps may be performed in turn or alternately.

Based on a same inventive concept, embodiments of the present disclosure further provide a coincidence event determination apparatus configured to implement the coincidence event determination method as referred to above. The implementation solution to the problem provided in the apparatus is similar to that described in the above method. Therefore, specific limitations in one or more embodiments of the coincidence event determination apparatus provided below may be obtained with reference to the limitations on the coincidence event determination method above. Details are not described herein again.

In an exemplary embodiment, as shown in FIG. 8, a coincidence event determination apparatus is provided, including: an energy window acquisition module 810, a detector determination module 820, and a coincidence event determination module 830.

The energy window acquisition module 810 is configured to acquire a first energy window and a second energy window that are asymmetrical.

The detector determination module 820 is configured to determine, in a case that a first detected energy satisfying the first energy window and a second detected energy satisfying the second energy window are detected, a first detector detecting the first detected energy and a second detector detecting the second detected energy.

The coincidence event determination module 830 is configured to determine the target coincidence event as a coincidence event corresponding to the first detector and the second detector.

In an exemplary embodiment, the first energy window includes a first LLD threshold, and the second energy window includes a second LLD threshold. The energy window acquisition module includes an LLD threshold unit, a first energy window unit, and a second energy window unit.

The LLD threshold unit is configured to acquire the first LLD threshold and the second LLD threshold that are different from each other. The first energy window unit is configured to obtain the first energy window based on the first LLD threshold. The second energy window unit is configured to obtain the second energy window based on the second LLD threshold.

In an exemplary embodiment, the detector determination module includes a first detected energy unit and a second detected energy unit.

The first detected energy unit is configured to take a detected energy greater than the first LLD threshold as the first detected energy. The second detected energy unit is configured to take a detected energy greater than the second LLD threshold as the second detected energy.

In an exemplary embodiment, the LLD threshold unit includes an initial threshold acquisition unit, an initial coincidence event acquisition unit, a standard coincidence event acquisition unit, and a threshold adjustment unit.

The initial threshold acquisition unit is configured to acquire a first low energy-level initial threshold and a second low energy-level initial threshold that are different from each other, and acquire a first low energy-level standard threshold and a second low energy-level standard threshold that are the same as each other. The initial coincidence event acquisition unit is configured to acquire an initial coincidence event determined according to the first low energy-level initial threshold and the second low energy-level initial threshold. The standard coincidence event acquisition unit is configured to acquire a standard coincidence event determined according to the first low energy-level standard threshold and the second low energy-level standard threshold. The threshold adjustment unit is configured to adjust the first low energy-level initial threshold and the second low energy-level initial threshold according to a degree of accuracy of the initial coincidence event and the standard coincidence event, to obtain the first LLD threshold and the second LLD threshold that are different from each other.

In an exemplary embodiment, the coincidence event determination module includes a target first detector unit, a target second detector unit, and a target coincidence event unit.

The target first detector unit is configured to acquire the first detector correspondingly detecting the first detected energy as a target first detector. The target second detector is configured to acquire a first detection time at which the first detected energy is detected, and determine the second detector that detects the second detected energy at a second detection time as a target second detector. The first detection time and the second detection time are times within a preset time window. The target coincidence event unit is configured to obtain the target coincidence event according to coincidence events detected by the target first detector and the target second detector.

In an exemplary embodiment, the coincidence event determination module includes a scattering correction coefficient determination unit and a target coincidence event determination unit.

The scattering correction coefficient determination unit is configured to perform Monte Carlo simulation on a preset simulated detection object to obtain a scattering correction coefficient. The target coincidence event determination unit is configured to correct the detected coincidence event according to the scattering correction coefficient, to obtain the corrected target coincidence event.

In an exemplary embodiment, the energy window acquisition module includes an asymmetrical energy window mode determination unit and an energy window determination unit.

The asymmetrical energy window mode determination unit is configured to acquire, in response to a trigger instruction of an asymmetrical energy window mode, the asymmetrical energy window mode. The energy window determination unit is configured to acquire the first energy window and the second energy window that are asymmetrical in a case that an imaging mode is the asymmetrical energy window mode.

The modules in the coincidence event determination apparatus and the photovoltaic charging system above may be implemented entirely or partially by software, hardware, or a combination thereof. The above modules may be built in or independent of a processor of a controller in a hardware form, or may be stored in a memory of the controller in a software form, to facilitate the processor to invoke and perform operations corresponding to the above modules.

In an embodiment, the present disclosure provides a PET image reconstruction method which may be used to perform step S104, i.e., the step of performing image reconstruction based on the PET raw data corresponding to the target coincidence event, to obtain a reconstructed image of the to-be-detected object. That is, step S104 may include steps S902 to S906 below.

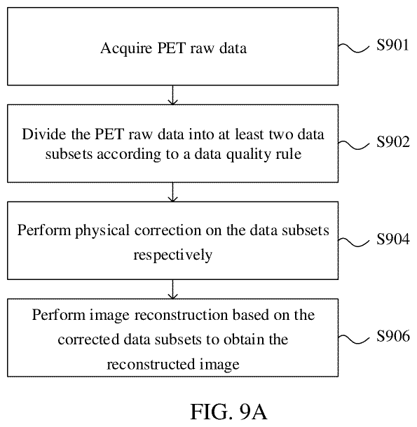

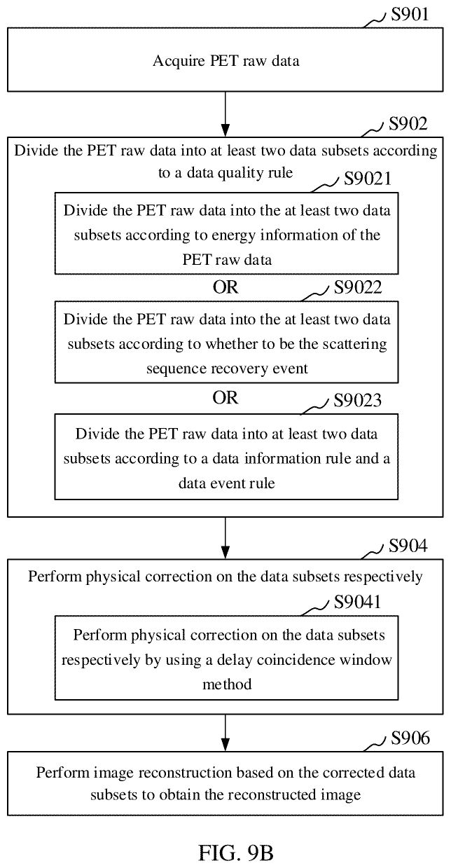

In an exemplary embodiment, as shown in FIG. 9A and FIG. 9B, the PET image reconstruction method includes S902 to S906 below.

In step S902, the PET raw data is divided into at least two data subsets according to a data quality rule.

Prior to step S902, step S901 of acquiring PET raw data may be further included. Step S901 of acquiring PET raw data may be implemented through steps S102 and S104 above. The PET raw data includes position information of a pair of detectors at two ends of the target coincidence event, and time of flight (TOF) information. During the PET detection, a radioactive nuclide is used to label a metabolite, and this labeled metabolite will emit a positron through decay emission, the positron annihilates with a surrounding electron to produce a pair of photons emitted in opposite directions. If the two photons are detected simultaneously by detector crystals of a PET detection system, then the nuclide is considered to be on a line connecting the corresponding pair of detectors (a group of detectors) that has captured the photons, and this line is called a line of response (LOR). A response line corresponds to a coincidence event. Therefore, a pair of detectors at two ends of the target coincidence event refers to a pair of detectors at two ends of a response line corresponding to the target coincidence event. A current PET reconstruction algorithm only divides the PET raw data according to different TOF information, but does not distinguish other information. The TOF information is an inaccurate time difference between photons reaching detectors at two ends of the LOR, which is information that assists in determining a position of a response line in space. The PET raw data is divided according to different TOF information, which is essentially a means of storing the PET raw data without affecting image quality. Therefore, in this embodiment, there is a need to further mine information carried in the PET raw data to improve the quality of PET image reconstruction.

The data quality rule can be information that characterizes the data characteristic of the PET raw data. The data quality rule may include a data information rule and a data event rule. The data information rule is attributes of the PET raw data, including energies, scattering degrees, and the like. The data event rule is a preset coincidence manner of the PET raw data, including whether the detectors at two ends of the target coincidence event coincide with a scattering sequence recovery event. For example, the data characteristic of the PET raw data can include at least energy information and/or event information. The data characteristic of the PET raw data can also include energy information and/or event information in combination with TOF information.

In step S904, physical correction is performed on the data subsets respectively.

Step S904 may include step S9041.

In step S9041, physical correction is performed on the data subsets respectively by using a delay coincidence window method.

It should be noted that other existing physical correction methods may alternatively be used to perform physical correction on the data subsets respectively, which is not limited to the delay coincidence window method.

In step S906, an image reconstruction is performed based on the corrected data subsets to obtain the reconstructed image.

For example, the image reconstruction may be performed based on the corrected data subsets and according to an image reconstruction iterative algorithm to obtain the reconstructed image.

The image reconstruction iterative algorithm includes an ordered subset maximum expectation reconstruction algorithm.

It should be noted that image reconstruction may alternatively be performed on corrected data subsets respectively by using other existing image reconstruction methods, which is not limited to the image reconstruction iterative algorithm.

In this implementation, the PET raw data corresponding to the target coincidence event is divided into at least two data subsets according to the data quality rule, and then physical correction and image reconstruction are respectively performed on data subsets of different qualities according to different characteristics of the data subsets. Image reconstruction is performed by using PET raw data of different qualities, which can improve utilization of the PET raw data and also help improve the quality of the reconstructed image. For example, the reconstructed image may have a higher contrast, clearer resolution, and the like.

In an exemplary embodiment, the data quality rule includes a data information rule, and the data information rule includes energy information and the like. Step S902 includes step S9021.

In step S9021, the PET raw data is divided into the at least two data subsets according to energy information of the PET raw data.

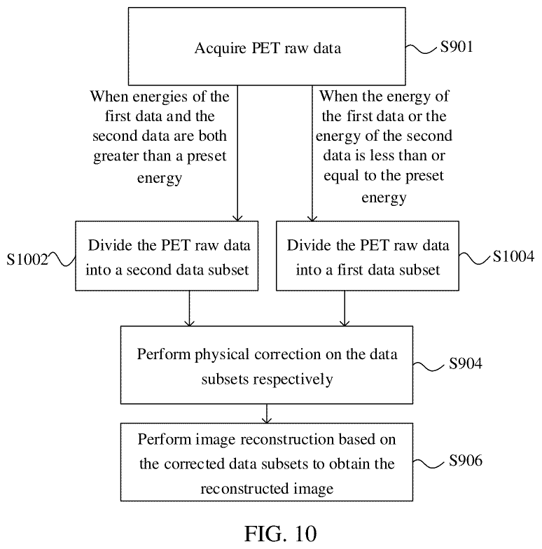

The PET raw data includes first data and second data obtained from the pair of detectors at the two ends of the target coincidence event. Referring to FIG. 10, step S9021 includes:

performing step S1002 when energies of the first data and the second data are both greater than a preset energy; and performing step S1004 when the energy of the first data or the energy of the second data is less than or equal to the preset energy.

In step S1002, the PET raw data is divided into a first data subset.

In step S1004, the PET raw data is divided into a second data subset.

It should be noted that the terms “first data”, “second data”, “first data subset”, and “second data subset” are all used for descriptive purposes only, which cannot be construed as indicating or implying a relative importance or implicitly specifying the number of the indicated technical features.

An energy of the first data subset is greater than that of the second data subset. The preset energy is set according to an actual situation. For example, if an energy range of the first data and the second data obtained from the pair of detectors at the two ends of the target coincidence event is 200 keV to 650 keV and the preset energy is 400 keV, the PET raw data with the first data and the second data whose energies are both greater than 400 keV and less than or equal to 650 keV is divided into a high-level first data subset. The PET raw data with the first data (or the second data) whose energy is greater than 200 keV and less than or equal to 400 keV is divided into a low-level second data subset.

In this embodiment, data subsets are classified based on the energy information of the PET raw data, and physical correction and image reconstruction are performed on different data subsets respectively, which helps speed up convergence of the image and improve accuracy of correction. In particular, a contrast of reconstruction of a cold-region image can be increased. The cold-region image means that an imaging agent is mainly taken up by functional normal tissues and is basically not taken up by diseased tissues, a static image shows morphology of normal tissues and organs, and lesion sites show sparse or defective radioactive distribution.

In an exemplary embodiment, the data quality rule includes a data event rule, and the data event rule includes a scattering sequence recovery event. Step S902 includes step S9022.

In step S9022, the PET raw data is divided into the at least two data subsets according to whether to be the scattering sequence recovery event.

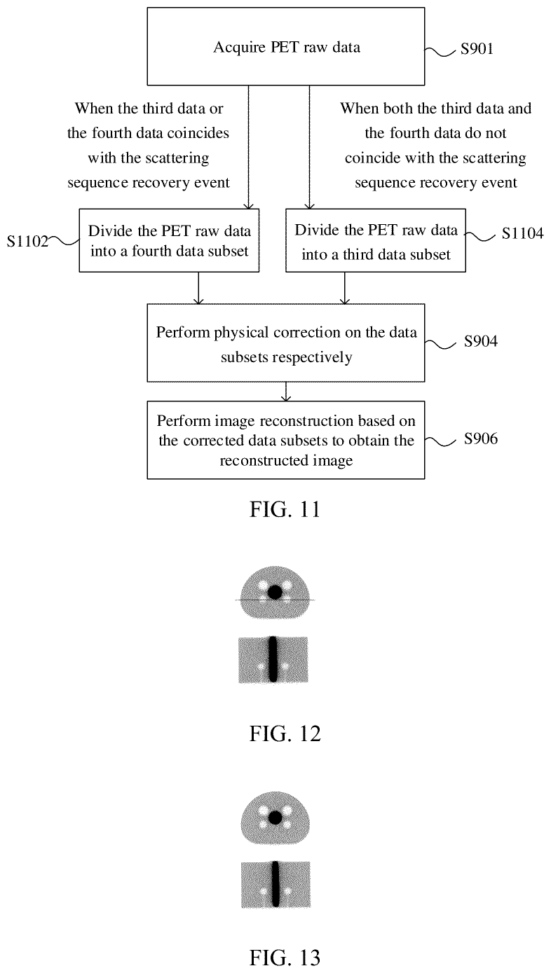

The PET raw data includes third data and fourth data obtained from the pair of detectors at the two ends of the target coincidence event. Referring to FIG. 11, step S9022 includes:

performing step S1102 when both the third data and the fourth data do not coincide with the scattering sequence recovery event; and performing step S1104 when the third data or the fourth data coincides with the scattering sequence recovery event.

In step S1102, the PET raw data is divided into a third data subset.

In step S1104, the PET raw data is divided into a fourth data subset.

It should be noted that the terms “third data”, “fourth data”, “third data subset”, and “fourth data subset” are all used for descriptive purposes only, which cannot be construed as indicating or implying a relative importance or implicitly specifying the number of the indicated technical features. In this implementation, the PET raw data is divided according to whether the third data and the fourth data obtained from the pair of detectors at the two ends of the target coincidence event coincides with the scattering sequence recovery event, to obtain the third data subset and the fourth data subset, and physical correction and image reconstruction are performed on the third data subset and the fourth data subset respectively, which helps improve image resolution. That is, under a same number of iterations, a full width at half maxima (FWHM) of a PET reconstructed point source data image is reduced, or a capability of the PET to distinguish adjacent point sources is improved.

In an exemplary embodiment, step S902 includes step S9023.

In step S9023, the PET raw data is divided into at least two data subsets according to a data information rule and a data event rule.

The data information rule includes other data information rules such as energy information. The data event rule includes other data event rules such as scattering sequence recovery events.

In this embodiment, a classification criterion for the data subsets may be multi-dimensional, including not only the data information rule but also the data event rule, so that image reconstruction can be performed more accurately by using different types of data subsets, thereby improving a final display effect of the reconstructed image.

In an exemplary embodiment, the step S906 of performing image reconstruction based on the corrected the data subsets and according to an image reconstruction iterative algorithm to obtain the reconstructed image of the to-be-detected object includes: performing modeling on each of the corrected data subsets to obtain modeling data of each of the data subsets; and performing a joint reconstruction on a combination of the modeled data of the data subsets to obtain the reconstructed image of the to-be-detected object.

In an exemplary embodiment, step S906 includes: inputting the corrected data subsets into the ordered subset maximum expectation reconstruction algorithm according to the following formula to obtain the reconstructed image:

$${λ}_{j}^{n + 1} = \frac{{λ}_{j}^{n}}{{\sum }_{i ∈ {I}_{s}} ( \frac{{S}_{i ,}^{′}}{{A}_{i} {N}_{i}^{′}} + \frac{{S}_{i ,}^{″}}{{A}_{i} {N}_{i}^{″}} )} {\sum }_{i , t ∈ {I}_{s}} [ {S}_{i , j , t}^{′} \frac{{p}_{i , t}^{′}}{{\sum }_{l} {S}_{i , l , t}^{′} {λ}_{l}^{n} + {A}_{i} {N}_{i}^{′} ( {s}_{i , t}^{′} + {r}_{i , t}^{′} )} + {S}_{i , j , t}^{″} \frac{{p}_{i , t}^{″}}{{\sum }_{l} {S}_{i , l , t}^{″} {λ}_{l}^{n} + {A}_{i} {N}_{i}^{″} ( {s}_{i , t}^{″} + {r}_{i , t}^{″} )} ]$$

The data subsets include a fifth data subset and a sixth data subset. λⱼⁿ⁺¹ is used to represent a j^{th} pixel value on a reconstructed image A after an n+1^{th} iteration process. n represents the number of iteration. λⱼⁿ is used to represent a j^{th} pixel value on the reconstructed image λ after an n^{th} iteration process, and j denotes an index of an actual updated pixel value. λₗⁿ is used to represent an l^{th} pixel value on the reconstructed image λ after the n^{th} iteration process, and l denotes an index of a related pixel on an image required to be traversed in an orthographic projection process. p′_{i,t} is used to represent data of a response line i in the fifth data subset in a time window t. p′_{i,t} is used to represent data of the response line i in the sixth data subset in the time window t. S′_{i,j,t} is used to represent a response matrix in which data in the fifth data subset maps a pixel j on the image to the response line i and the time window t. S′_{i,j,t} is used to represent a response matrix in which data in the sixth data subset maps the pixel j on the image to the response line i and the time window t. S′_{i,l,t} is used to represent a response matrix in which the data in the fifth data subset maps a pixel l on the image to the response line i and the time window t. S″_{i,l,t} is used to represent a response matrix in which the data in the sixth data subset maps the pixel l on the image to the response line i and the time window t. s′_{i,t} is used to represent scattering estimation of p′_{i,t}. s″_{i,t} is used to represent scattering estimation of p′_{i,t} is used to represent random event estimation of p′_{i,t}. r″_{i,t} is used to represent random event estimation of p″_{i,t}. Aᵢ is used to represent an attenuation correction coefficient. N′ᵢ is used to represent a normalized correction coefficient of the fifth data subset. N″ᵢ is used to represent a normalized correction coefficient of the sixth data subset.

It should be noted that the terms “fifth data subset” and “sixth data subset” are all used for descriptive purposes only, which cannot be construed as indicating or implying a relative importance or implicitly specifying the number of the indicated technical features.

In addition, when a number of data subsets into which the PET raw data is divided according to the data quality rule is greater than two, data, response matrices, scattering estimation, random event estimation, and normalized correction coefficients in other data subsets are acquired, and a final reconstructed image is obtained according to the above formula.

A specific example is introduced below to describe the image reconstruction process of this embodiment in detail.

Energies of the PET raw data that can be detected by the pair of detectors at the two ends of the target coincidence event generally meet requirements of energy windows E_{LLD} to E_{ULD}. According to the energy information, the energies of the PET raw data of the detectors at the two ends higher than a specific energy Eₛₑₚ are defined as a high-quality data subset, and other data is defined as a general-quality data subset. The high-quality data subset has a smaller scattering fraction and a higher signal-to-noise ratio relative to the general-quality data subset. E_{LLD}, E_{ULD}, and Eₛₑₚ are set according to an actual situation.

After physical correction is performed on the high-quality data subset and the general-quality data subset respectively, image reconstruction is performed through the following formula:

$${λ}_{j}^{n + 1} = \frac{{λ}_{j}^{n}}{{\sum }_{i ∈ {I}_{s}} ( \frac{{S}_{i , j , t}^{′}}{{A}_{i} {N}_{i}^{′}} + \frac{{S}_{i , j , t}^{″}}{{A}_{i} {N}_{i}^{″}} )} {\sum }_{i , t ∈ {I}_{s}} [ {S}_{i , j , t}^{′} \frac{{p}_{i , t}^{′}}{{\sum }_{l} {S}_{i , l , t}^{′} {λ}_{l}^{n} + {A}_{i} {N}_{i}^{′} ( {s}_{i , t}^{′} + {r}_{i , t}^{′} )} + {S}_{i , j , t}^{″} \frac{{p}_{i , t}^{″}}{{\sum }_{l} {S}_{i , l , t}^{″} {λ}_{l}^{n} + {A}_{i} {N}_{i}^{″} ( {s}_{i , t}^{″} + {r}_{i , t}^{″} )} ]$$

wherein the data subsets include a high-quality data subset and a general-quality data subset; λⱼⁿ⁺¹ is used to represent a j^{th} pixel value on a reconstructed image λ after an n+l^{th} iteration process; X is used to represent a j^{th} pixel value on the reconstructed image λ after an n^{th} iteration process, and j denotes an index of an actual updated pixel value; λₗⁿ is used to represent an l^{th} pixel value on the reconstructed image λ after the n^{th} iteration process, and l denotes an index of a related pixel on an image required to be traversed in an orthographic projection process; p′_{i,t} is used to represent data of a response line i in the high-quality data subset in a time window t; p′_{i,t} is used to represent data of the response line i in the general-quality data subset in the time window t; S′_{i,j,t} is used to represent a response matrix in which data in the high-quality data subset maps a pixel j on the image to the response line i and the time window t; S″_{i,j,t} is used to represent a response matrix in which data in the general-quality data subset maps the pixel j on the image to the response line i and the time window t; S′_{i,l,t} is used to represent a response matrix in which the data in the high-quality data subset maps a pixel l on the image to the response line i and the time window t; S″_{i,l,t} is used to represent a response matrix in which the data in the general-quality data subset maps the pixel l on the image to the response line i and the time window t; s′_{i,t} is used to represent scattering estimation of p′_{i,t}; s″_{i,t} is used to represent scattering estimation of p″_{i,t}. r′_{i,t} is used to represent random event estimation of p′_{i,t}; r″_{i,t} is used to represent random event estimation of p″_{i,t}; Aᵢ is used to represent an attenuation correction coefficient; N′ᵢ is used to represent a normalized correction coefficient of the high-quality data subset; and N″ᵢ is used to represent a normalized correction coefficient of the general-quality data subset.

It should be noted that since reconstructed spatial positions of different data subsets are consistent in definition, the attenuation correction coefficient Aᵢ may be shared. However, scattering estimation and random event estimation corresponding to different data subsets are required to be calculated respectively, and the normalized correction coefficients are also required to be distinguished. Existing methods may be used for scattering estimation, random event estimation, and normalized correction, such as a single scattering estimation method, a Monte Carlo scattering estimation method, and a delay coincidence random correction method.

The following is a formula of an existing ordered subset maximum expectation reconstruction algorithm:

$${λ}_{j}^{n + 1} = \frac{{λ}_{j}^{n}}{{\sum }_{i ∈ {I}_{s}} \frac{{S}_{i , j}}{{A}_{i} \cdot {N}_{i}}} {\sum }_{i , t ∈ {I}_{s}} {S}_{i , j , t} \frac{{p}_{i , t}}{{\sum }_{l} {S}_{i , l , t} {λ}_{l}^{n} + {A}_{i} {N}_{i} ( {s}_{i , t} + {r}_{i , t} )}$$

where λⱼⁿ and λⱼⁿ⁺¹ respectively represent values of a j^{th} pixel on a reconstructed image λ in n^{th} and n+1^{th} iteration processes, p_{i,t} denotes coincidence prompt data of LORi in TOFb in which is t, and S_{i,l,t} denotes a response matrix in which a pixel l on the image is mapped to LORi, t. Aᵢ and Nᵢ denote attenuation correction and normalized correction coefficients of LORi respectively, and s_{i,t} and r_{i,t} denote scattering estimation and random event estimation in p_{i,t} respectively.

As can be seen, compared with the existing ordered subset maximum expectation reconstruction algorithm formula, for the ordered subset maximum expectation reconstruction algorithm formula of the high-quality data subset and the general-quality data subset obtained by division:

$$\begin{matrix}{p}_{i , t} = {p}_{i , t}^{′} + {p}_{i , t}^{″} ; \\ {s}_{i , t} = {s}_{i , t}^{′} + {s}_{i , t}^{″} ; \\ {r}_{i , t} = {r}_{i , t}^{′} + {r}_{i , t}^{″} ; and \\ \frac{1}{{N}_{i}} = \frac{1}{{N}_{i}^{′}} + \frac{1}{{N}_{i}^{″}} .\end{matrix}$$

Further, for energy division, if TOF resolution of the two sets of data is inconsistent but spatial resolution information of the LOR remains unchanged,

$${\sum }_{i ∈ {I}_{s}} ( \frac{{s}_{i , j , t}^{″}}{{A}_{i} {N}_{i}^{′}} + \frac{{s}_{i , j , t}^{″}}{{A}_{i} {N}_{i}^{″}} )$$

may be simplified into calculation

$${\sum }_{i ∈ {I}_{s}} \frac{{s}_{i , j}}{{A}_{i} \cdot {N}_{i}}$$

prior to LOR separation, and the above formula may be simplified into:

$${λ}_{j}^{n + 1} = \frac{{λ}_{j}^{n}}{{\sum }_{i ∈ {I}_{s}} \frac{{S}_{i , j}}{{A}_{i} \cdot {N}_{i}}} {\sum }_{i , t ∈ {I}_{s}} [ {S}_{i , j , t}^{′} \frac{{p}_{i , t}^{′}}{{\sum }_{l} {S}_{i , l , t}^{′} {λ}_{l}^{n} + {A}_{i} {N}_{i}^{′} ( {s}_{i , t}^{′} + {r}_{i , t}^{′} )} + {S}_{i , j , t}^{″} \frac{{p}_{i , t}^{″}}{{\sum }_{l} {S}_{i , l , t}^{″} {λ}_{l}^{n} + {A}_{i} {N}_{i}^{″} ( {s}_{i , t}^{″} + {r}_{i , t}^{″} )} ]$$

Further, if the TOF resolution and the spatial resolution of the two sets of data are consistent, the formula is simplified into:

$${λ}_{j}^{n + 1} = \frac{{λ}_{j}^{n}}{{\sum }_{i ∈ {I}_{s}} \frac{{S}_{i , j}}{{A}_{i} \cdot {N}_{i}}} {\sum }_{i , t ∈ {I}_{s}} {S}_{i , j , t} [ \frac{{p}_{i , t}^{′}}{{\sum }_{l} {S}_{i , l , t} {λ}_{l}^{n} + {A}_{i} {N}_{i}^{′} ( {s}_{i , t}^{′} + {r}_{i , t}^{′} )} + \frac{{p}_{i , t}^{″}}{{\sum }_{l} {S}_{i , l , t} {λ}_{l}^{n} + {A}_{i} {N}_{i}^{″} ( {s}_{i , t}^{″} + {r}_{i , t}^{″} )} ]$$

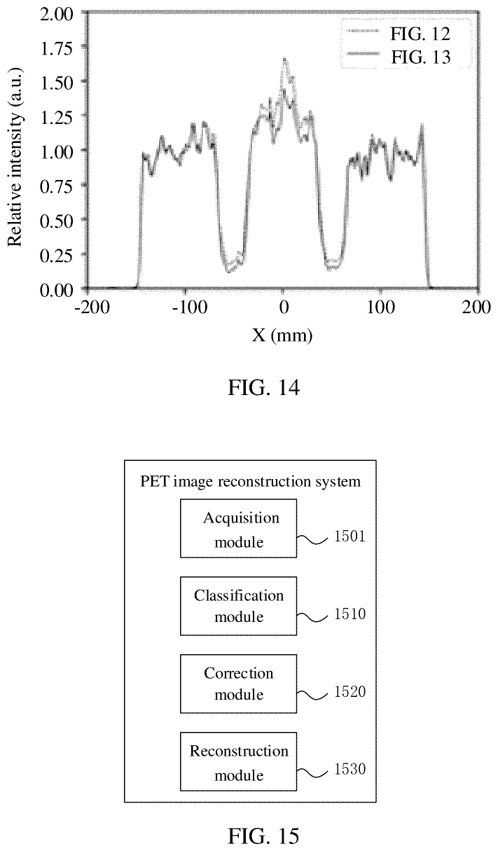

FIG. 12 shows a result of a reconstructed image in which the PET raw data is not divided into data subsets, and FIG. 13 is a result of a reconstructed image in which the PET raw data is divided into a high-quality data subset and a general-quality data subset in this example. FIG. 14 is a comparison diagram of relative intensity between the images in FIG. 12 and FIG. 13. In FIG. 14, the abscissa represents a position x of the PET detector that acquires the PET raw data, in units of mm, and the ordinate represents relative intensity, in an arbitrary unit (a.u.). In FIG. 14, the dotted line represents the image reconstruction result in FIG. 12, and the solid line represents the image reconstruction result in FIG. 13.

As can be seen from FIG. 12 to FIG. 14, the reconstructed image in which the PET raw data is divided into a high-quality data subset and a general-quality data subset has higher cold-region contrast and higher region border sharpness than the reconstructed image in which the PET raw data is not divided into data subsets.

In an exemplary embodiment, a PET image reconstruction system is provided. Referring to FIG. 15, the PET image reconstruction system includes:
a classification module 1510 configured to divide the PET raw data into at least two data subsets according to a data quality rule;
a correction module 1520 configured to perform physical correction on the data subsets respectively; and
a reconstruction module 1530 configured to perform image reconstruction based on the corrected data subsets to obtain the reconstructed image of the to-be-detected object.

The image reconstruction may be performed based on the corrected data subsets and according to an image reconstruction iterative algorithm to obtain the reconstructed image of the to-be-detected object. The image reconstruction iterative algorithm includes an ordered subset maximum expectation reconstruction algorithm.

In an exemplary embodiment, referring to FIG. 15, the PET image reconstruction system may further include an acquisition module 1501. The acquisition module 1501 is configured to acquire PET raw data. The step of acquiring PET raw data may include, for example, steps S102 and S104 above.

In an exemplary embodiment, the data quality rule includes a data information rule, and the data information rule includes energy information. The classification module 1510 is further configured to divide the PET raw data into at least two data subsets according to energy information of the PET raw data.

In an exemplary embodiment, the PET raw data includes first data and second data obtained from the pair of detectors at the two ends of the target coincidence event. The classification module 1510 is further configured to divide the PET raw data into a first data subset when energies of the first data and the second data are both greater than a preset energy; and further configured to divide the PET raw data into a second data subset when the energy of the first data or the energy of the second data is less than or equal to the preset energy.

An energy of the first data subset is greater than that of the second data subset.

In an exemplary embodiment, the data quality rule includes a data event rule, and the data event rule includes a scattering sequence recovery event. The classification module 1510 is further configured to divide the PET raw data into the at least two data subsets according to whether to be the scattering sequence recovery event.

In an exemplary embodiment, the PET raw data includes third data and fourth data obtained from the pair of detectors at the two ends of the target coincidence event. The classification module 1510 is further configured to divide the PET raw data into a third data subset when both the third data and the fourth data do not coincide with the scattering sequence recovery event; and divide the PET raw data into a fourth data subset when the third data or the fourth data coincides with the scattering sequence recovery event.

In an exemplary embodiment, the correction module 1520 is further configured to perform physical correction on the data subsets respectively by using a delay coincidence window method.

In an exemplary embodiment, the reconstruction module 1530 is further configured to input the corrected data subsets into the ordered subset maximum expectation reconstruction algorithm according to the following formula to obtain the reconstructed image:

$${λ}_{j}^{n + 1} = \frac{{λ}_{j}^{n}}{{\sum }_{i ∈ {I}_{s}} \frac{{s}_{i , j , t}^{′}}{{A}_{i} \cdot {N}_{i}^{′}} + \frac{{s}_{i , j , t}^{″}}{{A}_{i} \cdot {N}_{i}^{″}}} {\sum }_{i , t ∈ {I}_{s}} [ {S}_{i , j , t}^{′} \frac{{p}_{i , t}^{′}}{{\sum }_{l} {S}_{i , l , t}^{′} {λ}_{l}^{n} + {A}_{i} {N}_{i}^{′} ( {s}_{i , t}^{′} + {r}_{i , t}^{′} )} + {S}_{i , j , t}^{″} \frac{{p}_{i , t}^{″}}{{\sum }_{l} {s}_{i , l , t}^{″} {λ}_{l}^{n} + {A}_{i} {N}_{i}^{″} ( {s}_{i , t}^{″} + {r}_{i , t}^{″} )} ]$$

wherein the data subsets include a fifth data subset and a sixth data subset; λⱼⁿ⁺¹ is used to represent a j^{th} pixel value on a reconstructed image λ after an n+l^{th} iteration process; λⱼⁿ is used to represent a j^{th} pixel value on the reconstructed image λ after an n^{th} iteration process, and j denotes an index of an actual updated pixel value; λₗⁿ is used to represent an l^{th} pixel value on the reconstructed image λ after the n^{th} iteration process, and l denotes an index of a related pixel on an image required to be traversed in an orthographic projection process; p′_{i,t} is used to represent data of a response line i in the fifth data subset in a time window t; p′_{i,t} is used to represent data of the response line i in the sixth data subset in the time window t; S′_{i,j,t} is used to represent a response matrix in which data in the fifth data subset maps a pixel j on the image to the response line i and the time window t; S″_{i,j,t} is used to represent a response matrix in which data in the sixth data subset maps the pixel j on the image to the response line i and the time window t; S′_{i,l,t} is used to represent a response matrix in which the data in the fifth data subset maps a pixel l on the image to the response line i and the time window t; S′_{i,l,t} is used to represent a response matrix in which the data in the sixth data subset maps the pixel l on the image to the response line i and the time window t; s′_{i,t} is used to represent scattering estimation of p′_{i,t}; s′_{i,t} is used to represent scattering estimation of p′_{i,t}; r′_{i,t} is used to represent random event estimation of p′_{i,t}; r″_{i,t} is used to represent random event estimation of p′_{i,t}; Aᵢ is used to represent an attenuation correction coefficient; N′ᵢ is used to represent a normalized correction coefficient of the fifth data subset; and N″ᵢ is used to represent a normalized correction coefficient of the sixth data subset.

It should be noted that implementation principles and technical effects of the modules of the PET image reconstruction system in this embodiment may be obtained with reference to the PET image reconstruction method in the above embodiments, which are not described in detail herein again.

In an embodiment, the present disclosure provides a PET imaging method. The PET imaging method may use both the coincidence event determination method and the PET image reconstruction method described above to perform steps S102 and S104 respectively.

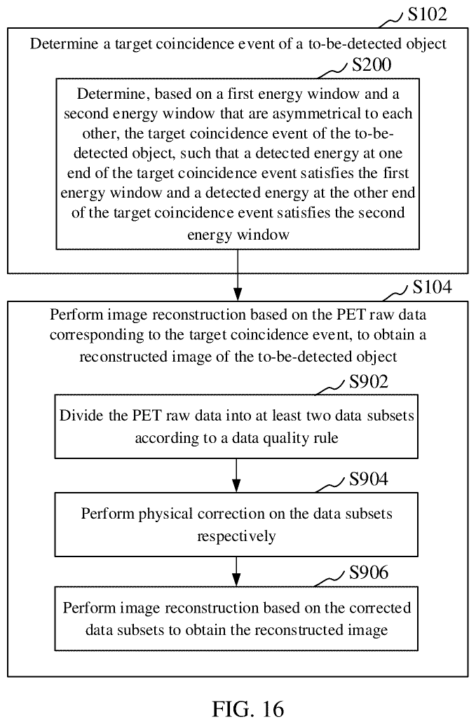

In an exemplary embodiment, as shown in FIG. 16, the PET imaging method includes S102 and S104 below.

In step S102, a target coincidence event of a to-be-detected object is determined.

Step S102 may include step S1022 of selectively determining the target coincidence event of the to-be-detected object, and step S1022 may include steps S200.

In step S200, based on a first energy window and a second energy window that are asymmetrical to each other, the target coincidence event of the to-be-detected object is determined such that a detected energy at one end of the target coincidence event satisfies the first energy window and a detected energy at the other end of the target coincidence event satisfies the second energy window.

In step S104, image reconstruction is performed based on the PET raw data corresponding to the target coincidence event, to obtain a reconstructed image of the to-be-detected object.

Step S104 may include steps S902 to S906.

In step S902, the PET raw data is divided into at least two data subsets according to a data quality rule.

In step S904, physical correction is performed on the data subsets respectively.

In step S906, an image reconstruction is performed based on the corrected data subsets to obtain the reconstructed image.

It should be noted that implementation details and technical effects of the steps in the PET imaging method in this embodiment may be obtained with reference to corresponding steps with the same numbers in the foregoing embodiments.

In an exemplary embodiment, a controller is provided. The controller may be a terminal, and a diagram of an internal structure thereof may be shown in FIG. 17. The controller includes a processor, a memory, an input/output (I/O) interface, a communication interface, a display unit, and an input apparatus. The processor, the memory, and the I/O interface are connected through a system bus. The communication interface, the display unit, and the input apparatus are connected to the system bus through the I/O interface. The processor of the controller is configured to provide computing and control capabilities. The memory of the controller includes a non-volatile storage medium and an internal memory. The non-volatile storage medium stores an operating system and a computer program. The internal memory provides an environment for running of the operating system and the computer program in the non-volatile storage medium. The I/O interface is configured to exchange information between the processor and an external device. The communication interface of the controller is configured to communicate with an external terminal in a wired or wireless manner. The wireless manner may be implemented by WIFI, a mobile cellular network, near field communication (NFC), or other technologies. The computer program is executed by the processor to implement the method described in the above embodiments of the present disclosure. The display unit of the controller is configured to form a visually visible picture, which may be a display screen, a projection apparatus, or a virtual reality imaging apparatus. The display screen may be a liquid crystal display screen or an electronic ink display screen. The input apparatus of the controller may be a touch layer covering the display screen, or may be a key, a trackball, or a touchpad disposed on a housing of the controller, or may be an external keyboard, a touchpad, a mouse, or the like.

Those skilled in the art may understand that the structure shown in FIG. 17 is only a block diagram of a partial structure related to a solution of the present disclosure, which does not constitute a limitation on the controller to which the solution of the present disclosure is applied. Specifically, the controller may include more or fewer components than those shown in the figure, or some components may be combined, or a different component deployment may be used.

In an exemplary embodiment, an electronic device 30 is provided. FIG. 18 is a schematic modular diagram of the electronic device 30. The electronic device 30 includes a memory, a processor, and a computer program stored in the memory and executable on the processor. The processor, when executing the program, implements the method described in the above embodiments. The electronic device 30 shown in FIG. 18 is merely an example, which should not be any limitation on the functions and scope of use of the embodiments of the present disclosure.

As shown in FIG. 18, the electronic device 30 may be in the form of a general computing device, which may be a server device, for example. Components of the electronic device 30 may include, but are not limited to, the at least one processor 31, the at least one memory 32, and a bus 33 connecting different system components (including the memory 32 and the processor 31).

The bus 33 includes a data bus, an address bus, and a control bus.

The memory 32 may include a volatile memory, such as a random access memory (RAM) 321 and/or a cache memory 322, and may further include a read-only memory (ROM) 323.

The memory 32 may further include a program/utility tool 325 having a group of (at least one) program modules 324. Such a program module 324 includes, but is not limited to, an operating system, one or more application programs, other program modules, and program data. Each or a combination of these examples may include implementation of a network environment.

The processor 31 runs the computer program stored in the memory 32, so as to execute various functional applications and data processing, for example, the method in the embodiments of the present disclosure.

The electronic device 30 may also communicate with one or more external devices 34 (for example, a keyboard, a pointing device, and the like). This communication may be performed through an I/O interface 35. Moreover, the electronic device 30 may also communicate with one or more networks (such as a local area network (LAN), a wide area network (WAN) and/or public networks, such as the Internet) through a network adapter 36. As shown in FIG. 18, the network adapter 36 communicates with other modules of the electronic device 30 through the bus 33. It should be understood that, although not shown in the figure, other hardware and/or software modules may be used with the electronic device 30, including, but not limited to, microcode, a device driver, a redundant processor, an external disk drive array, an redundant arrays of independent disks (RAID) system, a tape drive, a data backup storage system, and the like.

It should be noted that although several units/modules or sub-units/modules of the electronic device are mentioned in the foregoing detailed description, this division is merely an example and not mandatory. Actually, according to the implementations of the present disclosure, features and functions of two or more units/modules described above may be embodied in one unit/module. On the contrary, the features and functions of one unit/module described above may be further divided into a plurality of units/modules for reification.

In an exemplary embodiment, a non-transitory computer-readable storage medium is provided, having a computer program stored therein. The method described in the above embodiments is implemented when the program is executed by a processor.

More specifically, the readable storage medium may include, but is not limited to, a portable disk, a hard disk, a RAM, a ROM, an erasable programmable ROM, an optical storage device, a magnetic storage device, or any suitable combination thereof

In an exemplary embodiment, a computer program product is provided, including a program or program code. The method described in the above embodiments is implemented when the program or program code is executed by a processor.

Program codes for executing the present disclosure may be written in one programming language or a combination of multiple programming languages. The program codes may be executed entirely on a user device, executed partially on the user device, executed as a standalone software package, executed partially on the user device and partially on a remote device, or executed entirely on the remote device.

Those of ordinary skill in the art may understand that some or all procedures in the methods in the foregoing embodiments may be implemented by a computer program instructing related hardware, the computer program may be stored in a non-volatile computer-readable storage medium, and when the computer program is executed, the procedures in the foregoing method embodiments may be implemented. Any reference to the memory, storage, database, or other media used in the embodiments provided in the present disclosure may include at least one of a non-volatile memory and a volatile memory. The non-volatile memory may include a ROM, a magnetic tape, a floppy disk, a flash memory, an optical memory, a high-density embedded non-volatile memory, a resistive random access memory (ReRAM), a magnetoresistive random access memory (MRAM), a ferroelectric random access memory (FRAM), a phase change memory (PCM), a graphene memory, and the like. The volatile memory may include a random access memory (RAM) or an external cache. By way of illustration instead of limitation, the RAM is available in a variety of forms, such as a static random access memory (SRAM) or a dynamic random access memory (DRAM). The database as referred to in the embodiments provided in the present disclosure may include at least one of a relational database and a non-relational database. The non-relational database may include a blockchain-based distributed database, and the like, but is not limited thereto. The processor as referred to in the embodiments provided in the present disclosure may be a general-purpose processor, a central processing unit, a graphics processor, a digital signal processor, a programmable logic device, a data processing logic device based on quantum computing, and the like, but is not limited thereto.

The technical features in the above embodiments may be randomly combined. For concise description, not all possible combinations of the technical features in the above embodiments are described. However, all the combinations of the technical features are to be considered as falling within the scope described in this specification provided that they do not conflict with each other.

The above embodiments only describe several implementations of the present disclosure, and their description is specific and detailed, but cannot therefore be understood as a limitation on the patent scope of the present disclosure. It should be noted that those of ordinary skill in the art may further make variations and improvements without departing from the conception of the present disclosure, and these all fall within the protection scope of the present disclosure. Therefore, the patent protection scope of the present disclosure should be subject to the appended claims.

1. A positron emission computed tomography (PET) imaging method, comprising:
determining a target coincidence event of a to-be-detected object; and
performing image reconstruction based on PET raw data corresponding to the target coincidence event, to obtain a reconstructed image of the to-be-detected object.

2. The method according to claim 1, wherein determining the target coincidence event of the to-be-detected object comprises:
determining, based on a first energy window and a second energy window that are asymmetrical to each other, the target coincidence event of the to-be-detected object.

3. The method according to claim 2, wherein the first energy window comprises a low energy-level discriminator (LLD) threshold and/or a high energy-level discriminator (HLD) threshold, and the second energy window comprises a LLD threshold and/or a HLD threshold; and
wherein the LLD threshold and/or the HLD threshold of the first energy window is different from the LLD threshold and/or the HLD threshold of the second energy window.

4. The method according to claim 2, wherein the first energy window comprises a first low energy-level discriminator (LLD) threshold; the second energy window comprises a second LLD threshold; and
wherein the first LLD is different from the second LLD.

5. The method according to claim 4, wherein the first LLD threshold and the second LLD threshold are determined by the following process:
acquiring a first low energy-level initial threshold and a second low energy-level initial threshold;
adjusting the first low energy-level initial threshold and the second low energy-level initial threshold according to a preset condition, to obtain the first LLD threshold and the second LLD threshold that are different from each other.

6. The method according to claim 2, wherein the method further comprises:
obtaining a scattering correction coefficient; and
correcting the target coincidence event of the to-be-detected object according to the scattering correction coefficient.

7. The method according to claim 6, wherein the scattering correction coefficient is determined by performing Monte Carlo simulation on a simulated detection object.

8. The method according to claim 3, wherein the method further comprises:
performing, in response to a trigger instruction of an asymmetrical energy window mode, the step of determining the target coincidence event of the to-be-detected object based on the first energy window and the second energy window that are asymmetrical to each other.

9. The method according to claim 1, wherein performing image reconstruction based on the PET raw data corresponding to the target coincidence event, to obtain the reconstructed image of the to-be-detected object comprises:
dividing the PET raw data into at least two data subsets according to a data characteristic of the PET raw data;
performing physical correction on the data subsets respectively; and
performing image reconstruction based on the corrected data subsets to obtain the reconstructed image of the to-be-detected object.

10. The method according to claim 9, wherein the data characteristic of the PET raw data comprises one or a combination of the following:
energy information of the PET raw data;
event information of the PET raw data.

11. The method according to claim 9, wherein the data quality rule comprises a data information rule, the data information rule comprising energy information; and dividing the PET raw data into the at least two data subsets according to the data characteristic of the PET raw data comprises:
dividing the PET raw data into the at least two data subsets according to energy information of the PET raw data.

12. The method according to claim 11, wherein the PET raw data comprises first data and second data obtained from a pair of detectors at two ends of the target coincidence event; and dividing the PET raw data into the at least two data subsets according to the energy information of the PET raw data comprises:
dividing the PET raw data into a first data subset when energies of the first data and the second data are both greater than a preset energy; and
dividing the PET raw data into a second data subset when the energy of the first data or the energy of the second data is less than or equal to the preset energy.

13. The method according to claim 9, wherein dividing the PET raw data into the at least two data subsets according to the data characteristic of the PET raw data comprises:
dividing the PET raw data into the at least two data subsets according to whether to be the scattering sequence recovery event.

14. The method according to claim 13, wherein the PET raw data comprises third data and fourth data obtained from a pair of detectors at two ends of the target coincidence event; and dividing the PET raw data into the at least two data subsets according to whether to be the scattering sequence recovery event comprises:
dividing the PET raw data into a third data subset when both the third data and the fourth data do not coincide with the scattering sequence recovery event; and
dividing the PET raw data into a fourth data subset when the third data or the fourth data coincides with the scattering sequence recovery event.

15. The method according to claim 10, wherein performing physical correction on the data subsets respectively comprises:
performing physical correction on the data subsets respectively by using a delay coincidence window method.

16. The method according to claim 10, wherein performing image reconstruction based on the corrected data subsets to obtain the reconstructed image of the to-be-detected object comprises:
performing image reconstruction based on the corrected the data subsets and according to an image reconstruction iterative algorithm to obtain the reconstructed image of the to-be-detected object.

17. The method according to claim 16, wherein performing image reconstruction based on the corrected the data subsets and according to an image reconstruction iterative algorithm to obtain the reconstructed image of the to-be-detected object comprises:
performing modeling on each of the corrected data subsets to obtain modeling data of each of the data subsets; and
performing a joint reconstruction on a combination of the modeled data of the data subsets to obtain the reconstructed image of the to-be-detected object.

18. A PET imaging method, comprising:
determining a target coincidence event of a to-be-detected object, comprising:
determining, based on a first energy window and a second energy window that are asymmetrical to each other, the target coincidence event of the to-be-detected object; and
performing image reconstruction based on the PET raw data corresponding to the target coincidence event, to obtain a reconstructed image of the to-be-detected object, comprising:
dividing the PET raw data into at least two data subsets according to a data characteristic of the PET raw data;
performing physical correction on the data subsets respectively; and
performing image reconstruction based on the corrected data subsets to obtain the reconstructed image of the to-be-detected object.

19. An electronic device, comprising a memory and a processor, the memory storing a computer program, and the processor, when executing the computer program, implementing the PET imaging method according to claim 1.

20. A non-transitory computer-readable storage medium, having a computer program stored therein, wherein the PET imaging method according to claim 1 is implemented when the computer program is executed by a processor.
